(19) United States
(12) Patent Application Publication
(10) Pub. No.: US 2022/0184154 A1
(43) Pub. Date: Jun. 16, 2022

(54) TREATMENT OF BACTERIAL VAGINOSIS

(71) Applicants: Yeda Research and Development Co. Ltd., Rehovot (IL); Hadasit Medical Research Services and Development Ltd., Jerusalem (IL)

(72) Inventors: Eran ELINAV, Mazkeret Batya (IL); Yotam COHEN, Rehovot (IL); Ahinoam LEV-SAGIE, Lapid (IL); Debra GOLDMAN-WOHL, MaAle Adumim (IL)

(73) Assignees: Yeda Research and Development Co. Ltd., Rehovot (IL); Hadasit Medical Research Services and Development Ltd., Jerusalem (IL)

(21) Appl. No.: 17/681,946

(22) Filed: Feb. 28, 2022

Related U.S. Application Data
(63) Continuation of application No. PCT/IL2020/050937, filed on Aug. 27, 2020.

(30) Foreign Application Priority Data
Aug. 27, 2019 (IL) 268965

(51) Int. Cl. A61K 35/747; A61P 31/04

(52) U.S. Cl. CPC A61K 35/747; A61P 31/04

(57) ABSTRACT

A method of treating bacterial vaginosis in a subject is disclosed. The method comprises administering to the subject a therapeutically effective amount of a composition which comprises between three and twenty species of bacteria or a secretion thereof, wherein at least 70% of the bacteria of the composition are of the species Lactobacillus crispatus, thereby treating the bacterial vaginosis.

### RELATED APPLICATIONS

This application is a Continuation of PCT Patent Application No. PCT/IL2020/050937, having international filing date of Aug. 27, 2020 which claims the benefit of priority of Israel Patent Application No. 268965 filed on Aug. 27, 2019. The contents of the above applications are all incorporated by reference as if fully set forth herein in their entirety.

### SEQUENCE LISTING STATEMENT

The ASCII file, entitled 91047SequenceListing.txt, created on Feb. 28, 2022, comprising 44,266 bytes, submitted concurrently with the filing of this application is incorporated herein by reference.

### FIELD AND BACKGROUND OF THE INVENTION

The present invention, in some embodiments thereof, relates to methods and compositions for treating bacterial vaginosis.

Bacterial Vaginosis (BV) is considered the most prevalent form of vaginal infection in women of reproductive age, affecting from one-quarter to one-third of women. Women suffering from BV report diminished quality of life from symptoms that typically include abnormal, often malodorous vaginal discharge. Additionally, BV increases the risk of upper genital tract infection, complications of pregnancy, particularly preterm birth and lower success in fertility treatments, and of sexually transmitted infections (STI) including human immunodeficiency virus type 1 (HIV-1) infection, Herpes simplex virus type 2 (HSV-2), Chlamydia trachomatis, Neisseria gonorrhea, and Trichomonas vaginalis. Treatment with antibiotics (either systemic or vaginal) remains disappointing, with a high initial remission rate of 85%, complicated by a 30% relapse rate of symptomatic BV within three months of initial treatment, and up to 50-70% within a year. Many women therefore suffer from intractable, persistent or recurrent BV with very limited therapeutic options.

Treatment of recurrent BV consists of prolonged administration of oral or vaginal antibiotics, but even in transient responders, BV often relapses immediately after cessation of preventive treatment. Moreover, chronic or frequent antibiotic treatment, as recommended by Centers for Disease Control and Prevention (CDC) guidelines predisposes treated patients to the risk of vaginal candidiasis [17] and resistant infection. The economic impact of BV treatment is therefore exceedingly high, and is estimated in the USA alone to range around 3.7-6.1 billion dollars per year, excluding the costs of BV-associated pre-term birth and STI.

These alarming global health and economic implications of BV highlight the need to study the healthy vaginal microbiome and its mechanisms of providing vital microbial niche-specific functions. Indeed, in BV, the normal vaginal microbiome shifts from the usual protective microbial ecosystem dominated by Lactobacillus species to one characterized by the emergence of anaerobes [21-24]. Crucial to the pathogenesis of BV is the biofilm produced by anaerobes such as Gardnerella vaginalis, which adds to the difficulty in treatment and high recurrence rates of BV, due to poor local antibiotic penetration and activity [25]. Probiotic treatment with oral and/or vaginal administration of bacterial Lactobacillus strains has produced mixed results [26-27].

Background art includes WO2016100086A1; Dongwen Ma et al., FEMS Microbiology Letters 2019, Vol. 366, No. 4; and Clinical Trial (ClinicalTrials.gov.ID:NCT02236429, 2014).

Additional background art includes Cohen et al., New England Journal of Medicine 382:20, pages1906-1915 May 14, 2020.

### SUMMARY OF THE INVENTION

According to an aspect of the present invention there is provided a method of treating bacterial vaginosis in a subject in need thereof comprising administering to the subject a therapeutically effective amount of a composition which comprises between three and twenty species of bacteria or a secretion thereof, wherein at least 70% of the bacteria of the composition are of the species Lactobacillus crispatus and at least two more of the species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis and GCF-000787395 sp002021095, thereby treating the bacterial vaginosis.

According to an aspect of the present invention there is provided a composition comprising between three and twenty species of bacteria or a secretion thereof, wherein at least 70% of the bacteria of the composition are of the species Lactobacillus crispatus and at least two more of the species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis and GCF-000787395 sp002021095.

According to an aspect of the present invention there is provided a method of analyzing whether a composition is effective for treating a subject having BV, the method comprising:

(a) providing the composition to the subject; and

(b) analyzing the amount of at least one bacterial species in the vagina of the subject, the bacterial species being selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis, GCF-000787395 sp002021095, wherein an increase in the amount of the bacterial species following the providing as compared to the amount of the bacterial species prior to the providing is indicative that the composition was effective at treating the subject.

According to embodiments of the present invention, each of the at least two more of the species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis and GCF-000787395 sp002021095.

According to embodiments of the present invention, the composition comprises a cervico-vaginal secretion.

According to embodiments of the present invention, the composition comprises a conditioned medium of the bacteria.

According to embodiments of the present invention, the at least two more of the species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius and Bacteroides fragilis.

According to embodiments of the present invention, at least 1% of the bacteria are of the species Lactobacillus jensenii.

According to embodiments of the present invention, less than 10% of the bacteria of the composition are of the species Bifidobacterium vaginale.

According to embodiments of the present invention, the composition is comprised in a cervico-vaginal secretion from a donor subject who is not suffering from bacterial vaginosis.

According to embodiments of the present invention, the administering comprises transplanting to the vagina of the subject.

According to embodiments of the present invention, the bacteria of the composition are lyophilized or spray-dried.

According to embodiments of the present invention, the subject is Caucasian.

According to embodiments of the present invention, the bacterial vaginosis is intractable.

According to embodiments of the present invention, the composition is for use in treating bacterial vaginosis.

According to embodiments of the present invention, the method further comprises analyzing the amount of the bacterial species Bifidobacterium vaginale in the vagina of the subject, wherein a decrease in the amount of the bacterial species Bifidobacterium vaginale following the providing as compared to the amount of the bacterial species Bifidobacterium vaginale prior to the providing is indicative that the composition was effective at treating the subject.

According to embodiments of the present invention, the method further comprises analyzing the amount of Lactobacillus crispatus in the vagina of the subject, wherein an increase in the amount of Lactobacillus crispatus following the providing as compared to the amount of the bacterial species prior to the providing is indicative that the composition was effective at treating the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

### IN THE DRAWINGS

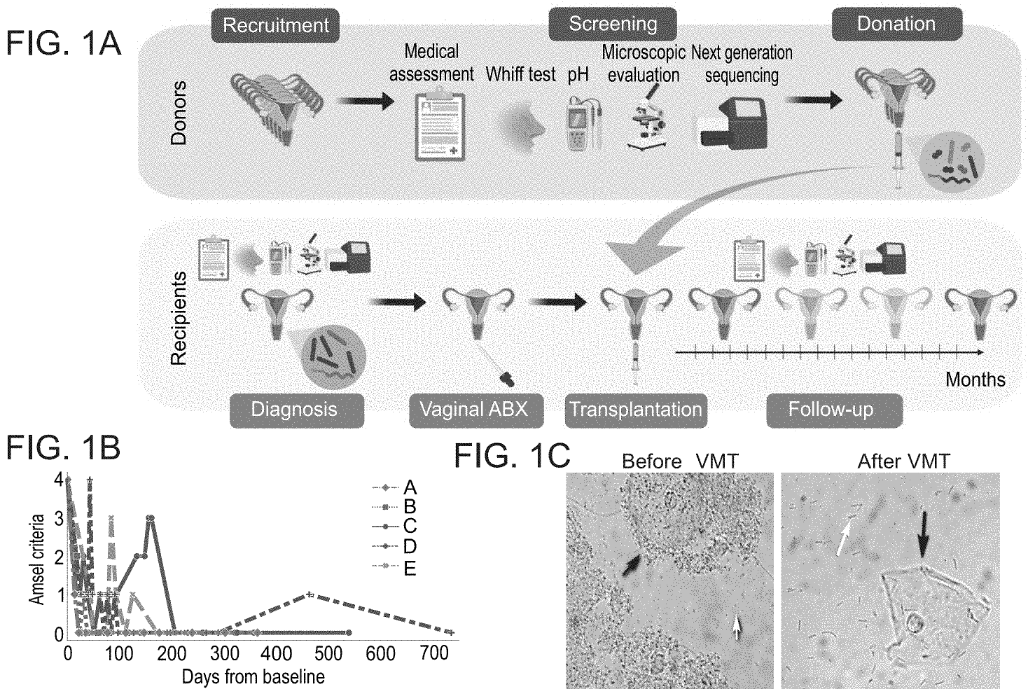

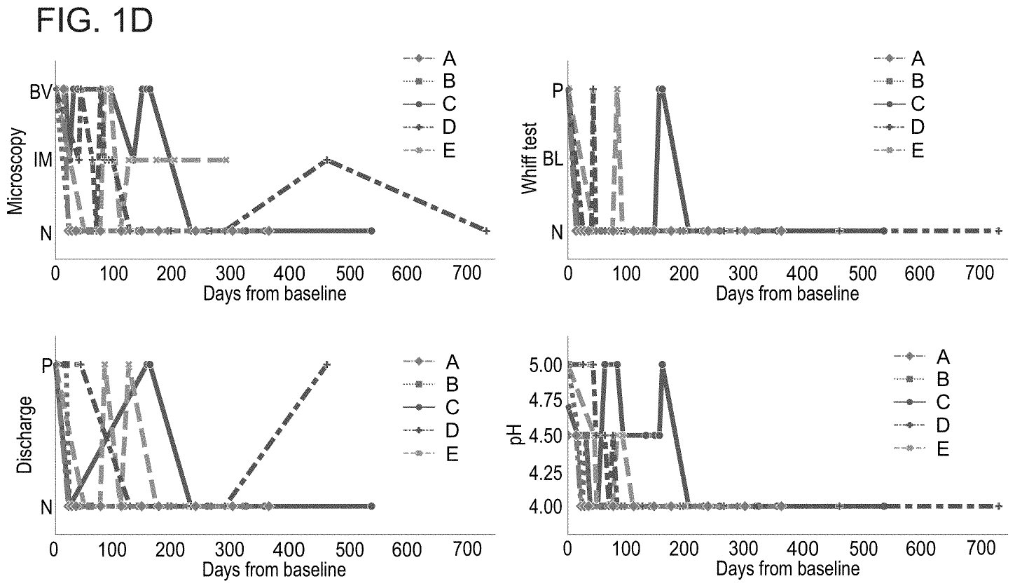

FIGS. 1A-D. Clinical features of VMT. (A) Schematic depiction of the VMT study; (B) Amsel's criteria (C) Wet mount microscopy before VMT: Clue cells (black arrow). The flora is comprised of abnormal coccid bacteria (white arrow). Wet mount microscopy after VMT: Normal, mature squamous epithelial cells (black arrow), and lactobacillus morphotypes (white arrow) are present. This wet mount represents normal vaginal discharge (original magnification ×400); (D) discrete clinical features, during post-VMT follow-up.

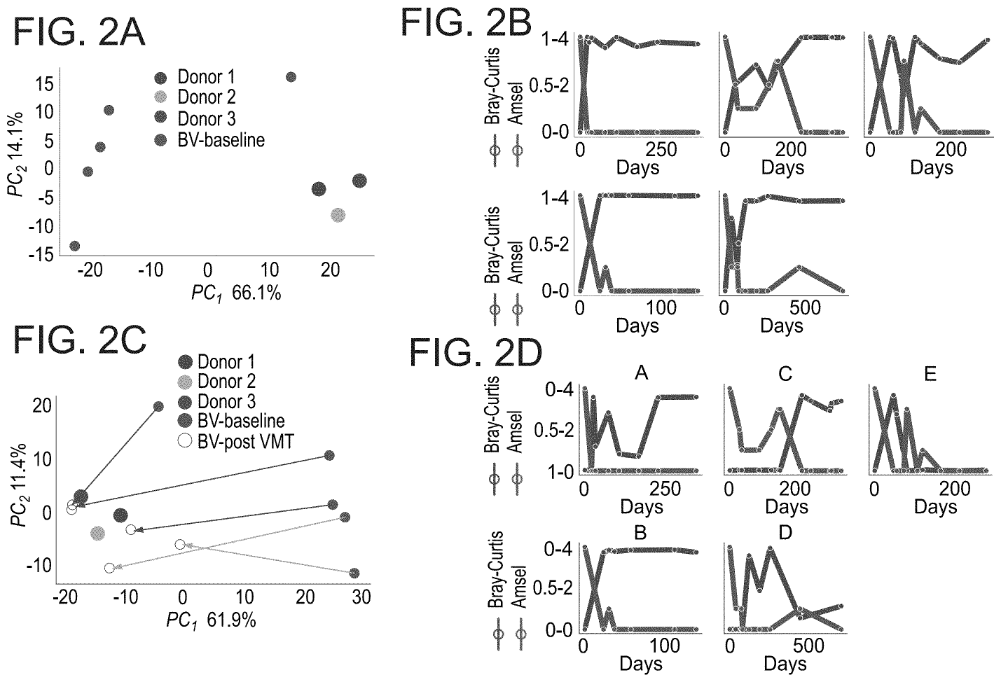

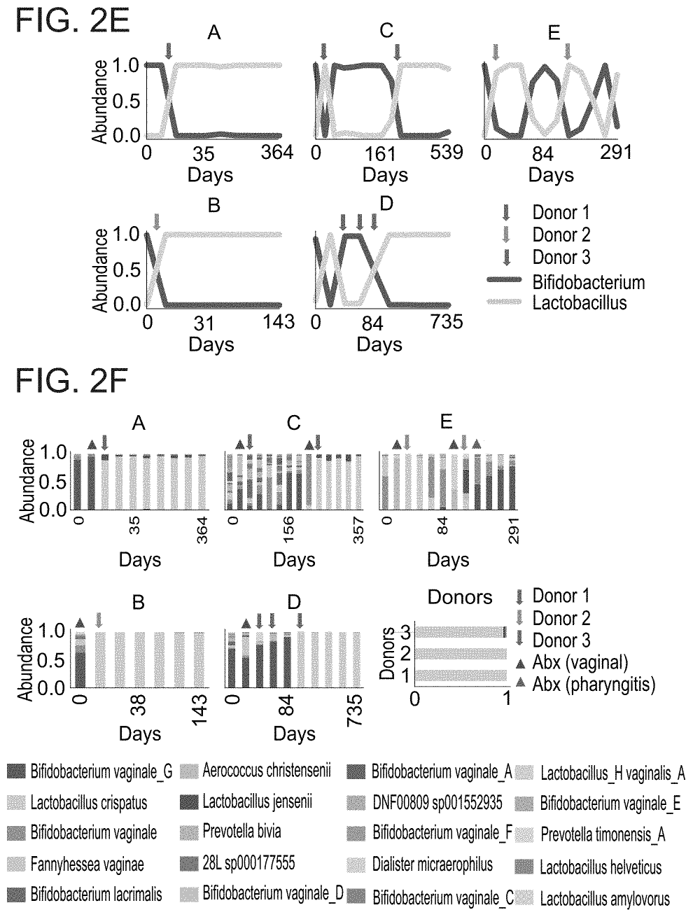

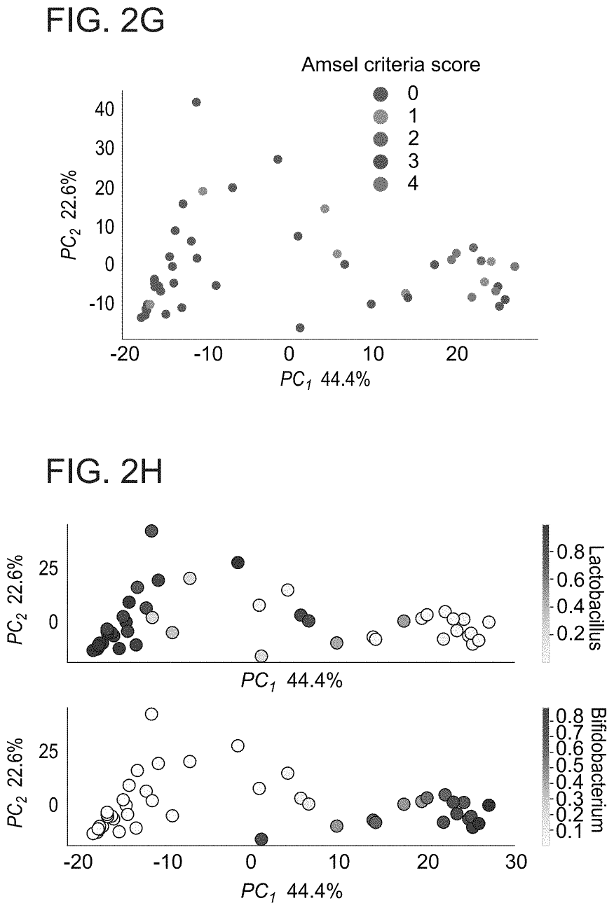

FIGS. 2A-H. Metagenomic microbiome assessment of the vaginal microbiome following VMT. (A) Metagenomic PCA performed on the donors and the recipients' baseline; (B) Metagenomic Bray-Curtis distance from baseline, correlated with the Amsel criteria scores; (C) Metagenomic PCA performed on the donors, baseline and last collected sample from each participant. Arrows depict the conversion of VMT recipients between baseline to post successful VMT, and are colored by the respective donor's color. Dots unconnected by the arrows represent the microbiome configuration of donors; (D) Metagenomic Bray-Curtis distance from respective donor, correlated with the Amsel criteria scores; (E) Metagenomic assessment of the change in the microbiome composition at the Genus level following VMT. Arrows indicate a VMT, their colors indicate the donor (F) Metagenomic bar plot denoting the species most contributing to the first PC. Arrows indicate a VMT, their colors indicate the donor, triangles indicate an antibiotic treatment (G,H) Metagenomic KEGG gene annotated principle component analysis, colored by G) Amsel criteria scored H) relative abundance of the Bifidobacterium genus and of the Lactobacillus genus.

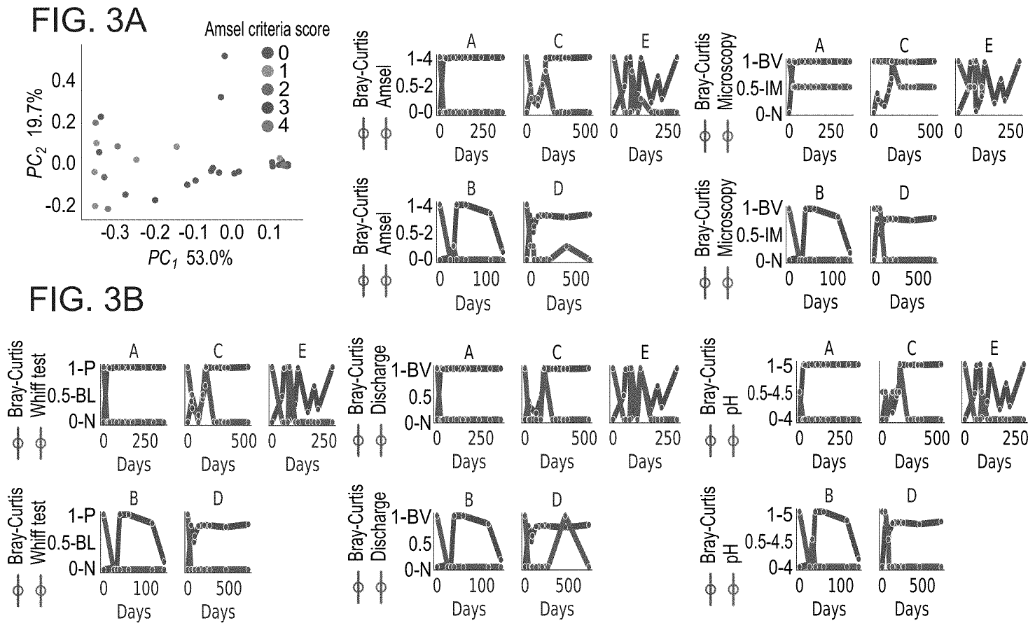

FIGS. 3A-B. 16s rDNA assessment of the vaginal microbiome following VMT. (A) 16S principle coordinates analysis using UniFrac distances colored by Amsel criteria scores. (B) Bray-Curtis distances from baseline, correlated with the Amsel criteria scores.

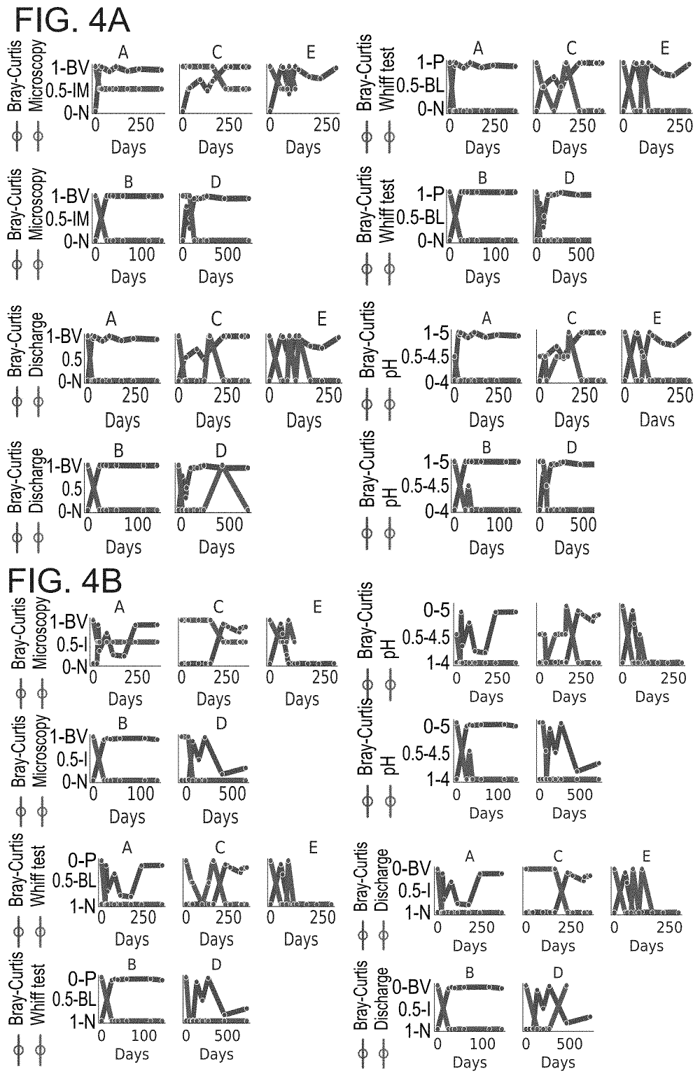

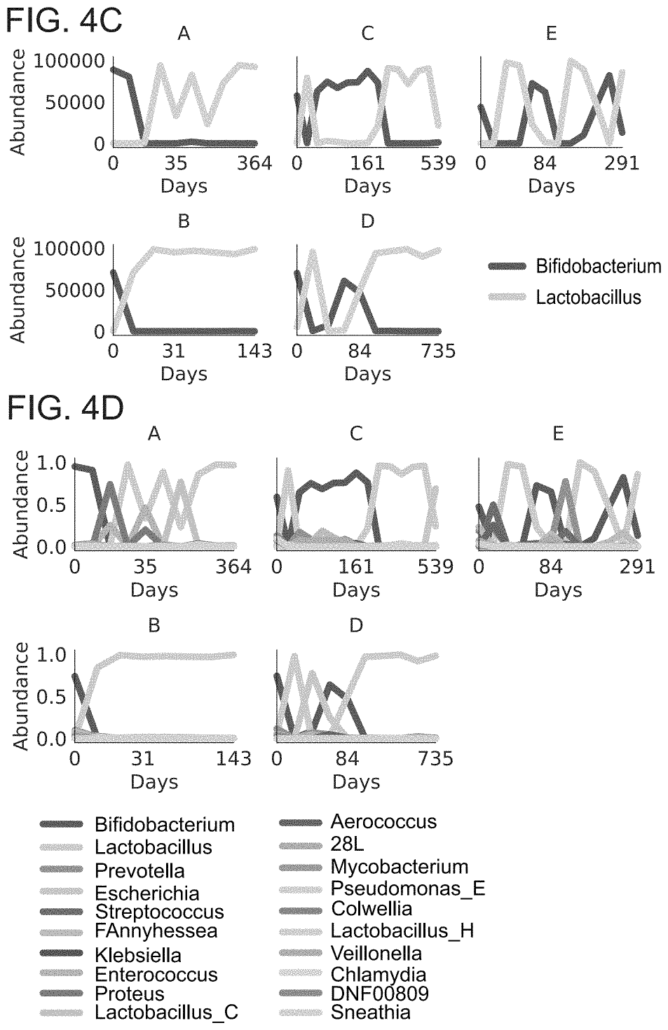

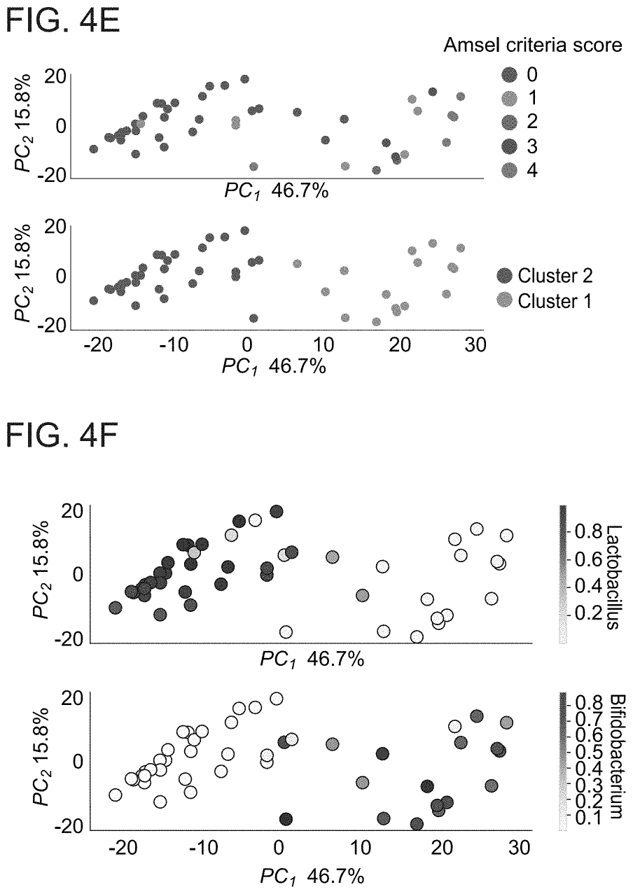

FIGS. 4A-F. Metagenomic compositional assessment of the vaginal microbiome following VMT. (A) Bray-Curtis distances from baseline, correlated with the Amsel criteria scores; (B) Bray-Curtis distances from respective donor, correlated with the Amsel criteria scores; (C) Metagenomic assessment of the change in the microbiome composition at the Genus level following VMT in absolute values; (D) Change in microbiome in the genus level following VMT; (E) PCA performed on the metagenomic taxonomic data colored by Amsel criteria scores and divided into cluster using 2-means algorithm; (F) PCA colored by relative abundance of Bifidobacterium and of Lactobacillus genus.

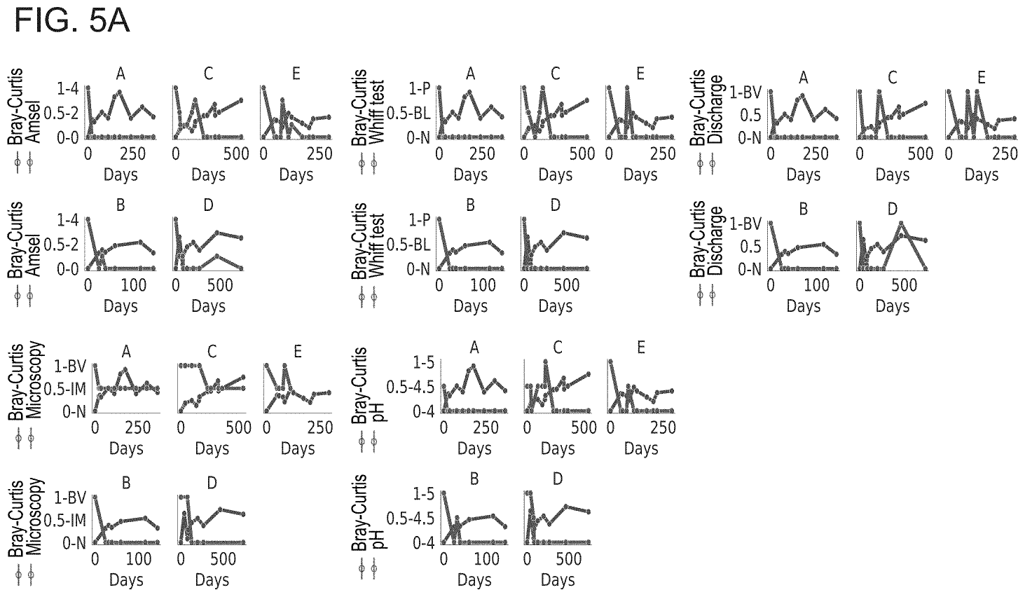

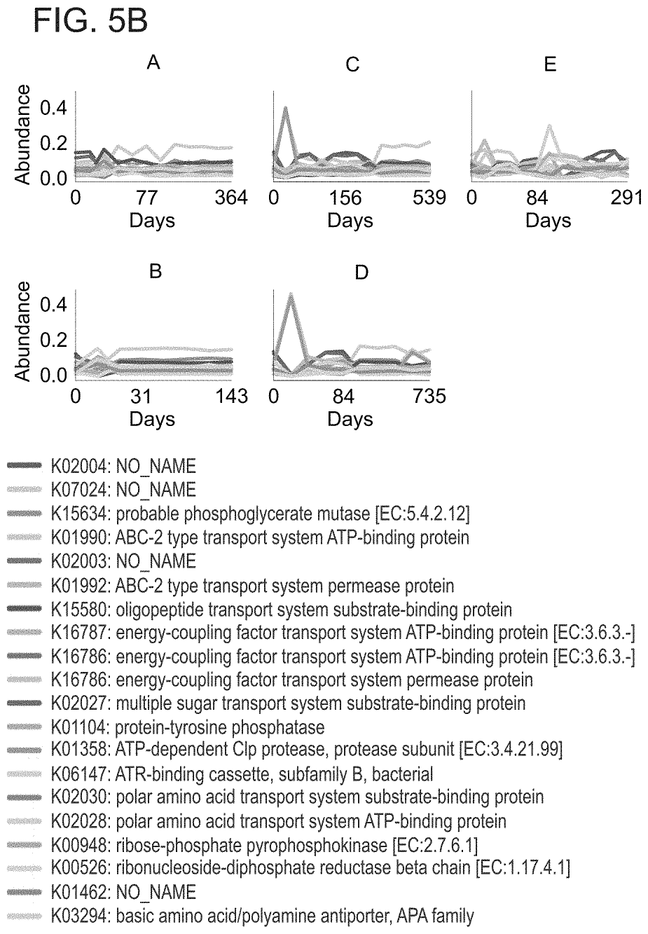

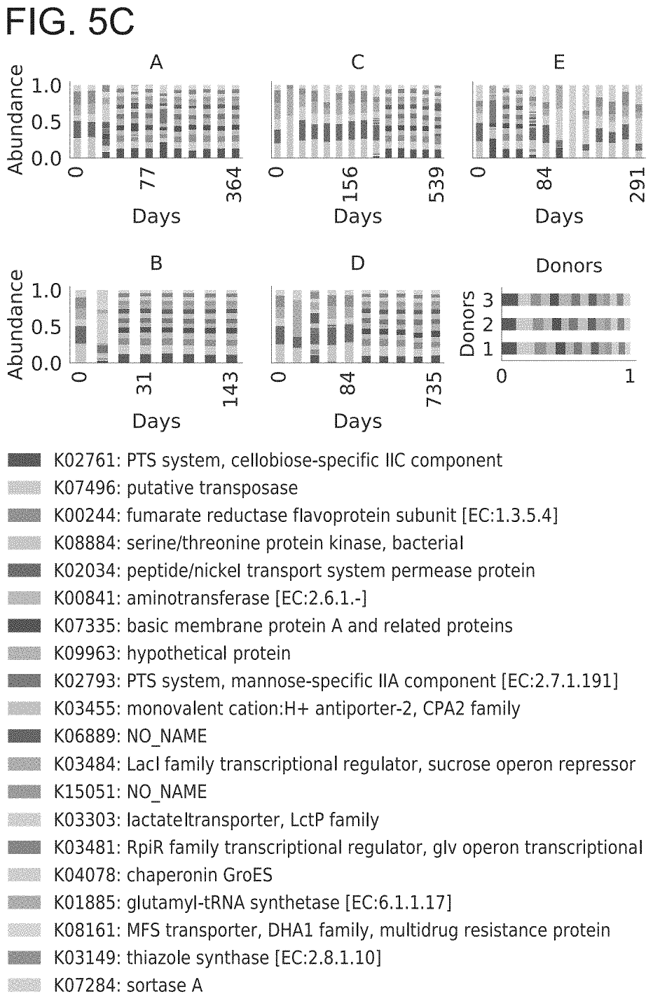

FIGS. 5A-C. Metagonomic functional (KEGG) assessment of the vaginal microbiome following VMT. (A) Bray-Curtis distances from baseline, correlated to Amsel criteria scores; (B) Change in microbiome functional KEGG gene annotated following VMT; D) Metagenomic bar plot denoting the KEGG genes that most contributed to the first principle component.

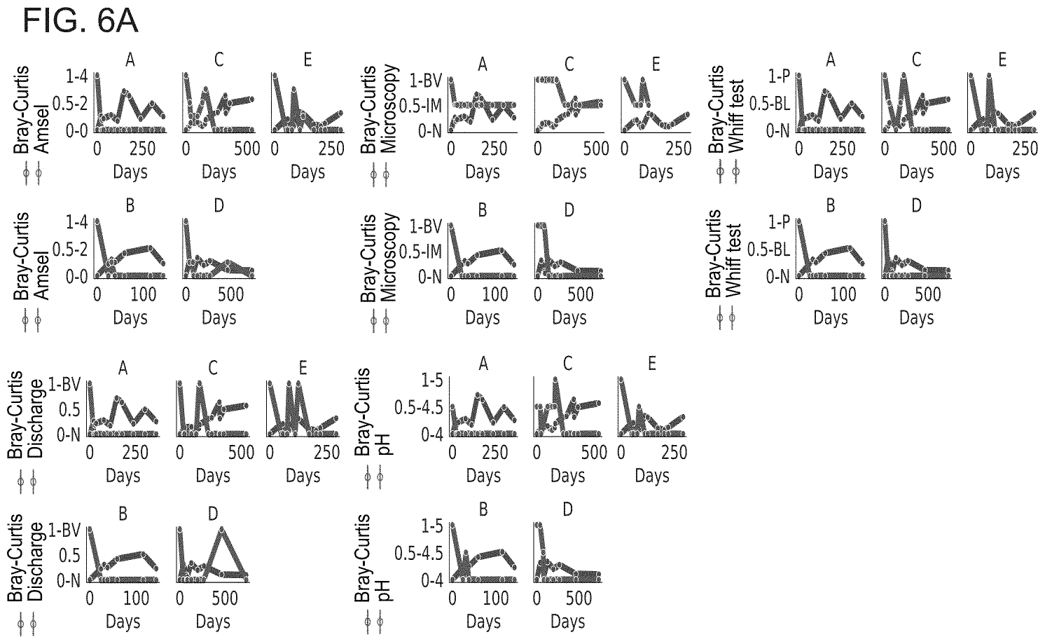

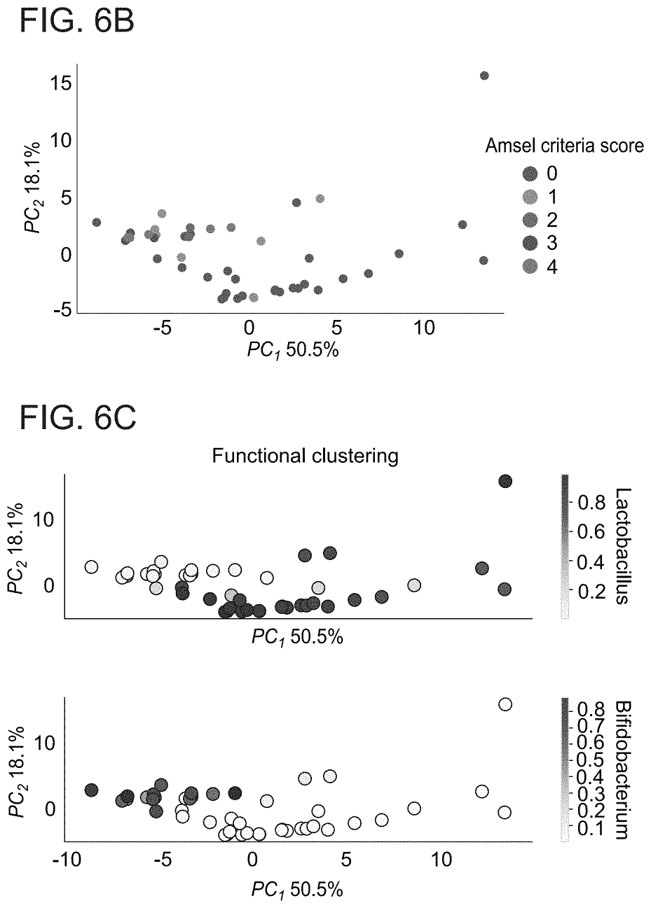

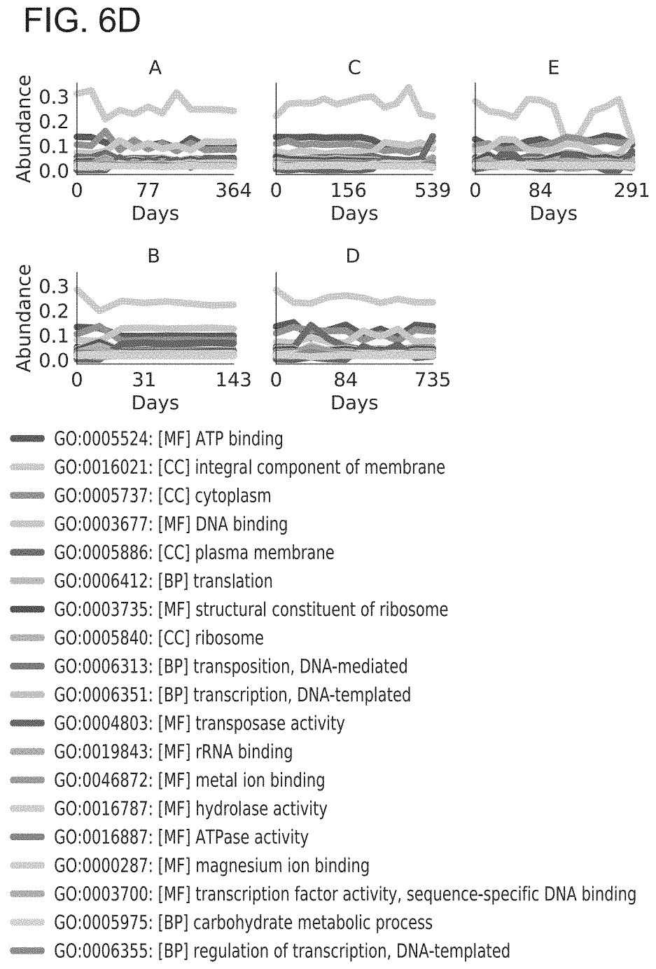

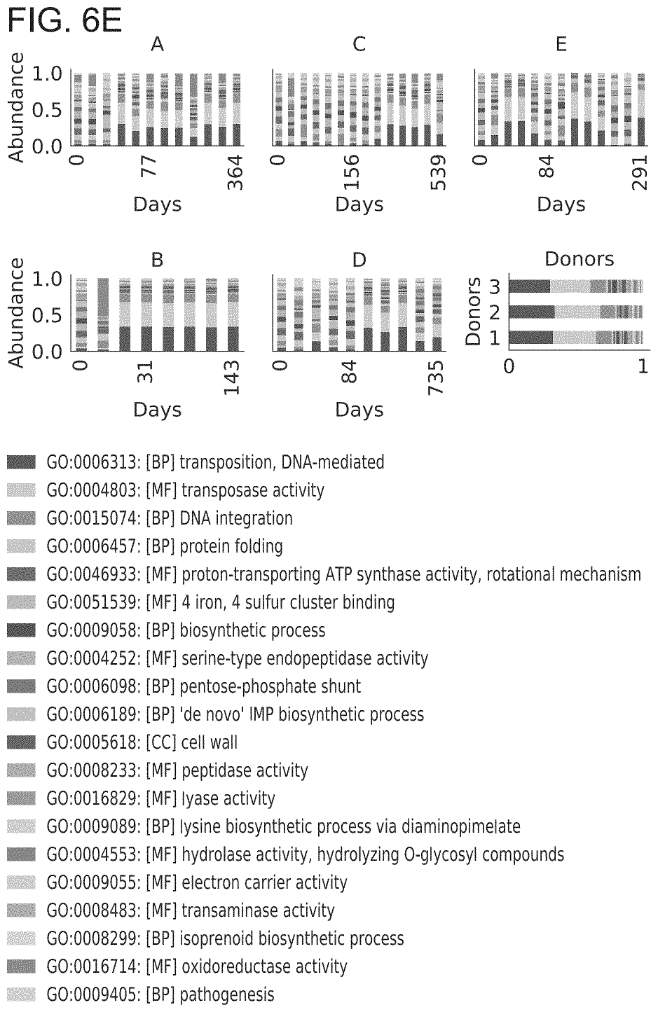

FIGS. 6A-E. Metagonomic functional (GO) assessment of the vaginal microbiome following VMT. (A) Bray-Curtis distances from baseline, correlated to Amsel criteria scores; (B,C) principle component analysis, colored by B) Amsel criteria score C) relative abundance of Bifidobacterium and of Lactobacillus genus; (D) Change in microbiome functional GO terms annotated following VMT; (E) Metagenomic bar plot denoting the GO terms that most contributed to the second principle component.

### DESCRIPTION OF SPECIFIC EMBODIMENTS OF THE INVENTION

The present invention, in some embodiments thereof, relates to methods and compositions for treating bacterial vaginosis.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Bacterial vaginosis (BV), caused by vaginal dysbiosis as well as the excessive growth of pathogenic bacteria, is a pathological condition of the vagina; its treatment using the antibiotics metronidazole or clindamycin often results in high recurrence rates. Considering the similar physiological environments of the intestinal tract and vaginal tract, as well as the pathological mechanism of intestinal infection and vaginal infection, the present inventors propose the conception of vaginal microbiota transplantation (VMT) for the long-term treatment of BV.

The present inventors effectively treated a number of patients suffering from recurrent BV by vaginal microbiome transplantation (VMT), taking the vaginal microbiome of a healthy donor and transplanting it into the patient. Whilst reducing the present invention to practice, the inventors found that a healthy vaginal microbiome, which is capable of bringing about a therapeutic effect in patients suffering from BV, is enriched with particular species of lactobacillus (namely Lactobacillus crispatus, Lactobacillus helveticus, Lactobacillus jensenii, Lactobacillus amylovorus, Lactobacillus gallinarum, Lactobacillus vaginalis). The present inventors propose that probiotic compositions enriched in these species or other species originating from healthy vaginal flora, as disclosed herein should be more effective at treating BV than single species compositions.

Thus, according to a first aspect of the present invention, there is provided a composition comprising between three and twenty species of bacteria or a secretion thereof, wherein at least 70% of the bacteria of the composition are of the species Lactobacillus crispatus and at least two more of said species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis, GCF-000787395 sp002021095.

According to a particular embodiment, the bacteria of the species Lactobacillus crispatus has a 16S rRNA nucleic acid sequence at least 95%, 96%, 97%, 98%, 99% identical to SEQ ID NO: 1.

According to a particular embodiment, the bacteria of the species Lactobacillus helveticus has a 16S rRNA nucleic acid sequence at least 95%, 96%, 97%, 98%, 99% identical to SEQ ID NO: 2.

According to a particular embodiment, the bacteria of the species Lactobacillus jensenii has a 16S rRNA nucleic acid sequence at least 95%, 96%, 97%, 98%, 99% identical to SEQ ID NO: 3.

According to a particular embodiment, the bacteria of the species Lactobacillus amylovorus has a 16S rRNA nucleic acid sequence at least 95%, 96%, 97%, 98%, 99% identical to SEQ ID NO: 4.

According to a particular embodiment, the bacteria of the species Lactobacillus gallinarum has a 16S rRNA nucleic acid sequence at least 90%, 91%, 92%, 93%, 94%, 95%, 96%, 97%, 98%, 99% identical to SEQ ID NO: 5.

According to a particular embodiment, the bacteria of the species Lactobacillus vaginalis has a 16S rRNA nucleic acid sequence at least 90%, 91%, 92%, 93%, 94%, 95%, 96%, 97%, 98%, 99% identical to SEQ ID NO: 6.

According to a particular embodiment, the bacteria of the species Mycobacterium sp001665295 has a 16S rRNA nucleic acid sequence at least 90%, 91%, 92%, 93%, 94%, 95%, 96%, 97%, 98%, 99% identical to SEQ ID NO: 7.

According to a particular embodiment, the bacteria of the species Paraburkholderia ginsengiterrae has a 16S rRNA nucleic acid sequence at least 90%, 91%, 92%, 93%, 94%, 95%, 96%, 97%, 98%, 99% identical to SEQ ID NO: 8.

According to a particular embodiment, the bacteria of the species Psychrobacter cibarius has a 16S rRNA nucleic acid sequence at least 90%, 91%, 92%, 93%, 94%, 95%, 96%, 97%, 98%, 99% identical to SEQ ID NO: 9.

According to a particular embodiment, the bacteria of the species Bacteroides fragilis has a 16S rRNA nucleic acid sequence at least 90%, 91%, 92%, 93%, 94%, 95%, 96%, 97%, 98%, 99% identical to SEQ ID NO: 10.

According to a particular embodiment, the bacteria of the species Pseudomonas fluorescens has a 16S rRNA nucleic acid sequence at least 90%, 91%, 92%, 93%, 94%, 95%, 96%, 97%, 98%, 99% identical to SEQ ID NO: 11.

According to a particular embodiment, the bacteria of the species GCF-000787395 sp002021095 has a 16S rRNA nucleic acid sequence at least 90%, 91%, 92%, 93%, 94%, 95%, 96%, 97%, 98%, 99% identical to SEQ ID NO: 12.

According to a particular embodiment, the bacteria of the species Alcanivorax hongdengensis has a 16S rRNA nucleic acid sequence at least 90%, 91%, 92%, 93%, 94%, 95%, 96%, 97%, 98%, 99% identical to SEQ ID NO: 13.

According to a particular embodiment, the bacteria of the species Colwellia echini has a 16S rRNA nucleic acid sequence at least 90%, 91%, 92%, 93%, 94%, 95%, 96%, 97%, 98%, 99% identical to SEQ ID NO: 14.

The bacteria may have a genomic sequence as defined by the accession numbers as set forth in Table 1:

TABLE 1

| | |
|---|---|
| Lactobacillus crispatus | GCF_002088015.1 |
| Lactobacillus helveticus | GCF_000160855.1 |
| Lactobacillus jensenii | GCF_001436455.1 |
| Lactobacillus amylovorus | GCF_002706375.1 |
| Lactobacillus gallinarum | GCF_000614735.1 |
| H vaginalis_A | GCF_003833155.1 |
| Mycobacterium sp001665295 | GCF_001665295.1 |
| Paraburkholderia ginsengiterrae | GCF_001645125.1 |
| Colwellia echini | GCF_002843355.1 |
| Psychrobacter cibarius | GCF_900016235.2 |
| Bacteroides fragilis_A | GCF_002849695.1 |
| Pseudomonas_E fluorescens_AN | GCF_001708445.1 |
| GCF-000787395 sp002021095 | GCF_002021095.1 |
| Alcanivorax hongdengensis | GCF_000300995.1 |

The bacteria may also be a variant of the bacteria defined by the above disclosed accession numbers, also referred to herein as a “functional homolog”.

The term “microbial strain” can refer to the strain having the above disclosed accession number and/or the functional homolog.

As used herein “functional homolog” or “functionally homologous” or “variant” or grammatical equivalents as used herein refers to a modification (i.e., at least one mutation) of the microbial strain (of the above disclosed accession number) resulting in a microbial strain that is endowed with substantially the same ensemble of biological activities (+/−10%, 20%, 40%, 50%, 60% when tested under the same conditions) as that of the deposited strain (e.g. therapeutic for the treatment of bacterial vaginosis) and can be classified to the same species or strain based on known methods of species/strain classifications.

Following are non-limiting criteria for identifying a functional homolog. These criteria, which are mostly genetic, combined with the functional characteristic of being therapeutic for bacterial vaginosis will be apparent to the skilled artisan define the scope of the functional homolog.

Thus, according to a specific embodiment, the deposited strain and the functional homolog belong to the same operational taxonomic units (OTU).

An “OTU” (or plural, “OTUs”) refers to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S-rRNA sequence or a portion of the 16S-rRNA (also referred to herein as “16S”) sequence or other functionally conserved genes as listed below. In other embodiments, the entire genomes of two entities are sequenced and compared. In another embodiment, select regions such as multilocus sequence tags (MLST, MLSA), specific genes, or sets of genes may be genetically compared. In 16S-rRNA embodiments, OTUs that share at least 97% average nucleotide identity across the entire 16S or some variable region of the 16S are considered the same OTU (see e.g. Claesson M J, Wang Q, O'Sullivan O, Greene-Diniz R, Cole J R, Ros R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940). In embodiments involving the complete genome, MLSTs, specific genes, or sets of genes OTUs that share at least 95% average nucleotide identity are considered the same OTU (see e.g. Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940). OTUs are frequently defined by comparing sequences between organisms. Such characterization employs, e.g., WGS data or a whole genome sequence.

According to a specific embodiment, the classification is based on DNA-DNA pairing data and/or sequence identity to functionally conserved genes or fragments thereof.

According to a specific embodiment a species/strain can be defined by DNA-DNA hybridization involving a pairwise comparison of two entire genomes and reflects the overall sequence similarity between them. According to a specific embodiment, a species is defined as a set of strains with at least about 70%, e.g., at least about 75%, at least about 80%, at least about 85%, at least about 90%, at least about 91%, at least about 92%, at least about 93%, at least about 94%, at least about 95% or more DNA-DNA relatedness and with 5 uC or less DTm.

According to a specific embodiment, the genomic nucleic acid sequence is at least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95% 99.95%, at least about 99.99%, at least about 99.999%, at least about 99.9999%, at least about 99.99999%, at least about 99.999999% or more DNA-DNA relatedness and with 5 uC or less DTm.

Thus, for example, if there is DNA-DNA hybridization on the basis of the article of Goris et al. [Goris, J., Konstantinidis, K. T., Klappenbach, J. A., Coenye, T., Vandamme, P., and Tiedje, J M. (2007). DNA-DNA hybridization values and their relationship to whole-genome sequence similarities. Int J Syst Evol Microbiol 57:81-91], some microorganisms expressing a DNA-DNA relatedness value of 70% or more (as described above) can be regarded as functional homologs according to some embodiments of the invention.

As used herein, “sequence identity” or “identity” or grammatical equivalents as used herein in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

Identity can be determined using any homology comparison software, including for example, the BlastN software of the National Center of Biotechnology Information (NCBI) such as by using default parameters.

According to some embodiments of the invention, the identity is a global identity, i.e., an identity over the entire nucleic acid sequences of the invention and not over portions thereof.

According to a specific embodiment, the genomic nucleic acid sequence is at least about 70%, e.g., at least 75%, at least about 80%, at least about 85%, at least about 90%, at least about 91%, at least about 92%, at least about 93%, at least about 94%, at least about 95%, at least about 96% least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95% 99.95%, at least about 99.99%, at least about 99.999%, at least about 99.9999%, at least about 99.99999%, at least about 99.999999% or more to the genomic sequence of the bacteria of the above disclosed accession numbers.

According to a specific embodiment, the genomic nucleic acid sequence is at least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, 99.95%, at least about 99.99%, at least about 99.999%, at least about 99.9999%, at least about 99.99999%, at least about 99.999999% or more identical to that of the above disclosed accession numbers.

According to an additional or alternative embodiment, a functional homolog is determined as the average nucleotide identity (ANI), which detects the DNA conservation of the core genome (Konstantinidis K and Tiedje J M, 2005, Proc. Natl. Acad. Sci. USA 102: 2567-2592). In some embodiments, the ANI between the functional homolog and the deposited strain is of at least about 95%, at least about, 96%, at least about 97%, at least about 98%, at least about 99%, at least about 99.1%, at least about 99.5%, at least about 99.6%, at least about 99.7%, at least about 99.8%, at least about 99.9%, at least about 99.99%, at least about 99.999%, at least about 99.9999%, at least about 99.99999%, at least about 99.999999% or more.

According to an additional or alternative embodiment, a functional homolog is determined by the degree of relatedness between the functional homolog and that of the bacteria of the above disclosed accession numbers determined as the Tetranucleotide Signature Frequency Correlation Coefficient, which is based on oligonucleotide frequencies (Bohlin J. et al. 2008, BMC Genomics, 9:104). In some embodiments, the Tetranucleotide Signature Frequency Correlation coefficient between the variant and those of the above disclosed accession numbers of about 0.99, 0.999, 0.9999, 0.99999, 0.999999, 0.999999 or more.

According to an additional or alternative embodiment, the degree of relatedness between the functional homolog and those of the above disclosed accession numbers is determined as the degree of similarity obtained when analyzing the genomes of the parent and of the variant strain by Pulsed-field gel electrophoresis (PFGE) using one or more restriction endonucleases. The degree of similarity obtained by PFGE can be measured by the Dice similarity coefficient. In some embodiments, the Dice similarity coefficient between the variant and the deposited strain is of at least about 96%, at least about 97%, at least about 98%, at least about 99%, at least about 99.1%, at least about 99.5%, at least about 99.6%, at least about 99.7%, at least about 99.8%, at least about 99.9%, at least about 99.99%, at least about 99.999%, at least about 99.9999%, at least about 99.99999%, at least about 99.999999% or more.

According to an additional or alternative embodiment, the functional homolog is defined as having the same ribotype, as obtained using any of the methods known in the art and described, for instance, by Bouchet et al. (Clin. Microbiol. Rev., 2008, 21:262-273).

According to an additional or alternative embodiment, the degree of relatedness between the functional homolog and those of the above disclosed accession numbers is determined by the Pearson correlation coefficient obtained by comparing the genetic profiles of both strains obtained by repetitive extragenic palindromic element-based PCR (REP-PCR) (see e.g. Chou and Wang, Int J Food Microbiol. 2006, 110:135-48). In some embodiments, the Pearson correlation coefficient obtained by comparing the REP-PCR profiles of the variant and the deposited strain is of at least about 0.99, at least about 0.999, at least about 0.9999, at least about 0.99999, at least about 0.999999, at least about 0.999999 or more.

According to an additional or alternative embodiment, the degree of relatedness between the functional homolog and those of the above disclosed accession numbers is defined by the linkage distance obtained by comparing the genetic profiles of both strains obtained by Multilocus sequence typing (MLST) (see e.g. Maiden, M. C., 1998, Proc. Natl. Acad. Sci. USA 95:3140-3145). In some embodiments, the linkage distance obtained by MLST of the functional homolog and the deposited strain is of at least about 0.99, at least about 0.999, at least about 0.9999, at least about 0.99999, at least about 0.999999, at least about 0.999999 or more.

According to an additional or alternative embodiment, the functional homolog comprises a functionally conserved gene or a fragment thereof e.g., a house-keeping gene e.g., 16S-rRNA or Internal Transcribed Spacer” (ITS), recA, glnII, atpD, gap, glnA, gltA, gyrB, pnp, rpoB, thrC or dnaK that is at least about 97%, at least about 98%, at least about 99%, at least about 99.1%, at least about 99.5%, at least about 99.6%, at least about 99.7%, at least about 99.8%, at least about 99.9%, at least about 99.99%, at least about 99.999%, at least about 99.9999%, at least about 99.99999%, at least about 99.999999% or more identical to those of the above disclosed accession numbers.

As mentioned, and according to a specific additional or an alternative embodiment, a functional homolog can also be determined on the basis of a multilocus sequence analysis (MLSA) determination of various functionally conserved genes or fragments thereof e.g., at least one, 2, 3, 4, 5, 6, 7, 8, 9, 10, 11, 12, 13, 14, 15 or more functionally conserved genes or fragments thereof, such as of e.g., 16S, ITS, recA, glnII, atpD, gap, glnA, gltA, gyrB, pnp, rpoB, thrC and dnaK.

According to a specific embodiment, the 16S ribosomal RNA (16S-rRNA) nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the ITS nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the recA nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the atpD nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the dnaK nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the glnII nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the gap nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the glnA nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the gltA nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the gyrB nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the pnp nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the rpoB nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to a specific embodiment, the thrC nucleic acid sequence is at least about 97%, e.g., at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95%, at least about 99.999%, at least about 99.9999%, at least about 99.99999%, at least about 99.999999% or more identical to those of the above disclosed accession numbers.

According to an additional or alternative embodiment the bacteria of the above disclosed accession numbers and the functional homolog is characterized by substantially the same (+/− about 10%, 20%, 40%, 50%, 60% when tested under the same conditions) biochemical profiling (e.g., biochemical fingerprinting) using for example, the GEN III redox chemistry (BIOLOG Inc. 21124 Cabot Blvd. Hayward Calif., 94545, USA), which can analyze both Gram-negative and Gram-positive bacteria, for their ability to metabolize all major classes of biochemicals, in addition to determining other important physiological properties such as pH, salt, and lactic acid tolerance. Further details can be obtained in “Modern Phenotypic Microbial Identification”, B. R. Bochner, Encyclopedia of Rapid Microbiological Methods, 2006, v. 2, Ch. 3, pp. 55-73, which is incorporated herein by reference in its entirety.

According to an additional or alternative embodiment, the functional homolog is defined by a comparison of coding sequences (gene) order.

According to an additional or alternative embodiment, the functional homolog is defined by a comparison of order of non-coding sequences.

According to an additional or alternative embodiment, the functional homolog is defined by a comparison of order of coding and non-coding sequences.

According to some embodiments of the invention, the combined coding region of the functional homolog is such that it maintains the original order of the coding regions as within the genomic sequence of the bacterial isolate yet without the non-coding regions.

For example, in case the genomic sequence has the following coding regions, A, B, C, D, E, F, G, each flanked by non-coding sequences (e.g., regulatory elements, introns and the like), the combined coding region will include a single nucleic acid sequence having the A+B+C+D+E+F+G coding regions combined together while maintaining the original order of their genome, yet without the non-coding sequences.

According to some embodiments of the invention, the combined non-coding region of the functional homolog is such that it maintains the original order of the non-coding regions as within the genomic sequence of the bacterial isolate yet without the coding regions as originally present in the bacterial of one of the above disclosed accession numbers.

According to some embodiments of the invention, the combined non-coding region and coding region (i.e., the genome) of the functional homolog is such that it maintains the original order of the coding and non-coding regions as within the genomic sequence of the bacteria of one of the above disclosed accession numbers.

As used herein “maintains” relate to at least about 90%, 91%, 92%, 93%, 94%, 95%, 96%, 97%, 98%, 99% or 100% is maintained as compared to the bacteria of the above disclosed accession numbers.

According to an additional or alternative embodiment, the functional homolog is defined by a comparison of gene content.

According to a specific embodiment, the functional homolog comprises a combined coding region where at least about 90%, at least about 91%, at least about 92%, at least about 93%, at least about 94%, at least about 95%, at least about 96%, at least about 97%, at least about 98%, at least about 99%, or more (e.g., 100%) is identical to a combined coding region existing in genome of one of the bacteria of the above disclosed accession numbers.

As used herein “combined coding region” refers to a nucleic acid sequence including all of the coding regions of the bacteria of the above defined accession number yet without the non-coding regions of the bacteria of the above defined accession number.

According to an additional or alternative embodiment, the functional homolog is defined by a comparison of nucleotide composition and codon usage.

According to an additional or alternative embodiment, the functional homolog is defined by a method based on random genome fragments and DNA microarray technology. These methods are of sufficiently high resolution to for strain-to-species level identification.

One of ordinary skill in the art, based on knowledge of the classification criteria would know how to identify strains that are considered functional homologs.

An additional and more detailed description of species-to-strain classification can be found in:

Cho and Tiedje 2001 Bacterial species determination from DNA-DNA hybridization by using genome fragments and DNA microarrays;

Coenye et al. 2005 Towards a parokaryotic genomic taxonomy. FEMS Microbiol. Rev. 29:147-167;

Konstantinidis and Tiedje (2005) Genomic insights that advance the species definition for prokaryotes. Proc. Natl. Acad. Sci. USA 102:189-197;

Konstantinidis et al. 2006 Toward a more robust assessment of intraspecies diversity using fewer genetic markers. Appl. Environ. Microbiol. 72:7286-7293.

It is to be understood that one or more methods as described herein can be used to identify a functional homolog.

Genomic data can be obtained by methods which are well known in the art e.g., DNA sequencing, bioinformatics, electrophoresis, an enzyme-based mismatch detection assay and a hybridization assay such as PCR, RT-PCR, RNase protection, in-situ hybridization, primer extension, Southern blot, Northern Blot and dot blot analysis.

In one embodiment, the composition of this aspect of the present invention is a probiotic composition.

As used herein, the phrase “probiotic composition” refers to a composition which comprises live micro-organisms, which when administered in adequate amounts, confer a health benefit on the host. Probiotics are typically alive when administered, have viability and reproducibility based on in vivo testing, and during use and storage.

In one embodiment, at least 20%, at least 30%, at least 40%, at least 50%, at least 60%, at least 70%, at least 80%, at least 90%, at least 95% of the bacteria of the composition are viable.

According to a specific embodiment, the functional homolog and the deposited strain belong to the same genus.

According to a specific embodiment, the functional homolog and the deposited strain belong to the same species.

According to a specific embodiment, the functional homolog and the deposited strain belong to the same sub-species.

As used herein “preparation” refers to an isolate of bacteria in which the prevalence (i.e., concentration) of the microbial stain or functional homolog is enriched over that (exceeds that) found in nature. In nature, the microbial strain is typically part of the plant microbiome, consisting of more than thousands microbial species. According to some embodiments of the invention, the preparation comprises less than 50, 20, 10, 9, 8, 7, 6, 5, 4 microbial species, e.g., bacteria and fungi.

According to a specific embodiment, the microbial preparations comprises 1, 2, 3, 4, 5, 6, 7, 8, 9 or 10 microbial species.

According to a specific embodiment, the preparation comprises the microbial strain at a level of purity of at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95% or more, say 100% pure.

According to a specific embodiment, the preparation comprises the microbial strain at a level of purity of at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.95%, at least about 99.99%, at least about 99.99%, at least about 99.999% or more, say 100% pure.

According to a specific embodiment, the microbial strain comprises viable microbial cells (capable of replicating).

According to a specific embodiment, the microbial strain comprises sporulating microbes.

A “spore” or “spores” refers to microbes that are generally viable, more resistant to environmental influences such as heat and bactericidal or fungicidal agents than other forms of the same microbial species, and typically capable of germination and out-growth. Bacteria and fungi that are “capable of forming spores” are those bacteria and fungi comprising the genes and other necessary abilities to produce spores under suitable environmental conditions.

As used herein “enriched” refers to 2-10,000,000 fold enrichment over that found in nature in an isolate of a vaginal microbiome of a healthy subject comprising the disclosed bacteria or a functional homolog of same.

As used in here, the phrase “CFUs” or “Colony Forming Units” refers to the number of microbial cells in a defined sample (e.g. milliliter of liquid, square centimeter of surface, one seed of grain, etc.) that form colonies and thereafter numbered, on a semi-solid bacteriological growth medium.

According to specific embodiments, the enrichment in the composition e.g., preparation is 10² CFUs-10⁹ CFUs/seed or 10² CFUs-10⁹ CFUs/gr powder or 10² CFUs-10⁹ CFUs/ml.

According to specific embodiments, the enrichment in the composition e.g., preparation is 10² CFUs-10⁸ CFUs/seed or 10² CFUs-10⁸ CFUs/gr powder or 10² CFUs-10⁸ CFUs/ml.

According to specific embodiments, the enrichment in the composition e.g., preparation is 10² CFUs-10⁷ CFUs/seed or 10² CFUs-10⁷ CFUs/gr powder or 10² CFUs-10⁷ CFUs/ml.

According to specific embodiments, the enrichment in the composition e.g., preparation is 10² CFUs-10⁶ CFUs/seed or 10² CFUs-10⁶ CFUs/gr powder or 10² CFUs-10⁶ CFUs/ml.

According to specific embodiments, the enrichment in the composition e.g., preparation is 10² CFUs-10⁵ CFUs/seed or 10² CFUs-10⁵ CFUs/gr powder or 10² CFUs-10⁵ CFUs/ml.

According to specific embodiments, the enrichment in the composition e.g., preparation is 10² CFUs-10⁴ CFUs/seed or 10² CFUs-10⁴ CFUs/gr powder or 10² CFUs-10⁴ CFUs/ml.

According to specific embodiments, the enrichment in the composition e.g., preparation is 10² CFUs-10³ CFUs/seed or 10² CFUs-10³ CFUs/gr powder or 10² CFUs-10³ CFUs/ml.

According to specific embodiments, the enrichment in the composition e.g., preparation is 10³ CFUs-10⁹ CFUs/seed or 10³ CFUs-10⁹ CFUs/gr powder or 10³ CFUs-10⁹ CFUs/ml.

According to specific embodiments, the enrichment in the composition e.g., preparation, formulation, coated seed is 10⁴ CFUs-10⁹ CFUs/seed or 10⁴ CFUs-10⁹ CFUs/gr powder or 10⁴ CFUs-10⁹ CFUs/ml.

According to specific embodiments, the enrichment in the composition e.g., preparation, is 10⁵ CFUs-10⁹ CFUs/seed or 10⁵ CFUs-10⁹ CFUs/gr powder or 10⁵ CFUs-10⁹ CFUs/ml.

According to specific embodiments, the enrichment in the composition e.g., preparation is 10⁶ CFUs-10⁹ CFUs/seed or 10⁶ CFUs-10⁹ CFUs/gr powder or 10⁶ CFUs-10⁹ CFUs/ml.

According to specific embodiments, the enrichment in the composition e.g., preparation is 10⁷ CFUs-10⁹ CFUs/seed or 10⁷ CFUs-10⁹ CFUs/gr powder or 10⁷ CFUs-10⁹ CFUs/ml.

According to specific embodiments, the enrichment in the composition e.g., preparation is 10⁸ CFUs-10⁹ CFUs/seed or 10⁸ CFUs-10⁹ CFUs/gr powder or 10⁸ CFUs-10⁹ CFUs/ml.

According to specific embodiments, the enrichment in the composition e.g., preparation is 10⁸ CFUs-10⁹ CFUs/seed or 10⁸ CFUs-10⁹ CFUs/gr powder or 10⁸ CFUs-10⁹ CFUs/ml.

According to a specific embodiment the preparation comprises at least about 100 CFU or spores, at least about 10² CFUs/seed CFUs/gr or CFUs/ml, at least about 10² CFUs/seed CFUs/gr or CFUs/ml, at least about 10³ CFUs/seed CFUs/gr or CFUs/ml, at least about 10⁴ CFUs/seed CFUs/gr or CFUs/ml, at least about 10⁵ CFUs/seed CFUs/gr or CFUs/ml, at least about 10⁶ CFUs/seed CFUs/gr or CFUs/ml, at least about 10⁷ CFUs/seed CFUs/gr or CFUs/ml, at least about 10⁸ CFUs/seed CFUs/gr or CFUs/ml, at least about 10⁹ CFUs/gr or CFUs/ml.

The compositions of this aspect of the present invention may contain 3, 4, 5, 6, 7, 8, 9, 10, 11, 12 13, 14, 15, 16, 17, 18, 19 or 20 different species of bacteria.

In another aspect of the present invention there is provided a composition comprising between three and forty species of bacteria or a secretion thereof, wherein at least 70% of the bacteria of the composition are of the species Lactobacillus crispatus and at least two more of said species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis, GCF-000787395 sp002021095

The compositions of this aspect of the present invention may contain 3, 4, 5, 6, 7, 8, 9, 10, 11, 12, 13, 14, 15, 16, 17, 18, 19 or 20, 21, 22, 23, 24, 25, 26, 27, 28, 29 30, 31, 32, 33, 34, 35, 36, 37, 38, 39 or even 40 different species of bacteria.

The compositions described herein are enriched in the bacterial genus lactobacillus.

Lactobacillus is a genus of Gram-positive, facultative anaerobic or microaerophilic, rod-shaped, non-spore-forming bacteria. They are a major part of the lactic acid bacteria group (i.e., they convert sugars to lactic acid).

Examples of species of the lactobacillus genus include, but are not limited to: Lactobacillus acetotolerans, Lactobacillus acidifarinae, Lactobacillus acidipiscis, Lactobacillus acidophilus, Lactobacillus agilis, Lactobacillus algidus, Lactobacillus alimentarius, Lactobacillus allii, Lactobacillus alvei, Lactobacillus alvi, Lactobacillus amylolyticus, Lactobacillus amylophilus, Lactobacillus amylotrophicus, Lactobacillus amylovorus, Lactobacillus animalis, Lactobacillus animata, Lactobacillus antri, Lactobacillus apinorum, Lactobacillus apis, Lactobacillus apodemi, Lactobacillus aquaticus, Lactobacillus aviarius, Lactobacillus backii, Lactobacillus bambusae, Lactobacillus bifermentans, Lactobacillus bombi, Lactobacillus bombicola, Lactobacillus brantae, Lactobacillus brevis, Lactobacillus brevisimilis, Lactobacillus buchneri, Lactobacillus cacaonum, Lactobacillus camelliae, Lactobacillus capillatus, Lactobacillus casei group, Lactobacillus chiayiensis, Lactobacillus paracasei, Lactobacillus catenefornis, Lactobacillus caviae, Lactobacillus cerevisiae, Lactobacillus ceti, Lactobacillus coleohominis, Lactobacillus colini, Lactobacillus collinoides, Lactobacillus composti, Lactobacillus concavus, Lactobacillus coryniformis, Lactobacillus crispatus, Lactobacillus crustorum, Lactobacillus curieae, Lactobacillus curtus, Lactobacillus curvatus, Lactobacillus delbrueckii, Lactobacillus dextrinicus, Lactobacillus diolivorans, Lactobacillus equi, Lactobacillus equicursoris, Lactobacillus equigenerosi, Lactobacillus fabifermentans, Lactobacillus faecis, Lactobacillus faeni, Lactobacillus farciminis, Lactobacillus farraginis, Lactobacillus fermentum, Lactobacillus floricola, Lactobacillus florum, Lactobacillus formosensis, Lactobacillus fornicalis, Lactobacillus fructivorans, Lactobacillus frumenti, Lactobacillus fuchuencis, Lactobacillus furfuricola, Lactobacillus futsaii, Lactobacillus gallinarum, Lactobacillus gasseri, Lactobacillus gastricus, Lactobacillus ghanensis, Lactobacillus gigeriorum, Lactobacillus ginsenosidimutans, Lactobacillus gorillae, Lactobacillus graminis, Lactobacillus guizhouensis, Lactobacillus halophilus, Lactobacillus hammesii, Lactobacillus hamsteri, Lactobacillus harbinensis, Lactobacillus hayakitensis, Lactobacillus heilongjiangensis, Lactobacillus helsingborgensis, Lactobacillus helveticus, Lactobacillus herbarum, Lactobacillus heterohiochii, Lactobacillus hilgardii, Lactobacillus hokkaidonensis, Lactobacillus hominis, Lactobacillus homohiochii, Lactobacillus hordei, Lactobacillus iatae, Lactobacillus iners, Lactobacillus ingluviei, Lactobacillus insectis, Lactobacillus insicii, Lactobacillus intermedius, Lactobacillus intestinalis, Lactobacillus iwatensis, Lactobacillus ixorae, Lactobacillus japonicas, Lactobacillus jensenii, Lactobacillus johnsonii, Lactobacillus kalixensis, Lactobacillus kefiranofacien, Lactobacillus kefiri, Lactobacillus kimbladii, Lactobacillus kimchicus, Lactobacillus kimchiensis, Lactobacillus kisonensis, Lactobacillus kitasutonis, Lactobacillus koreensis, Lactobacillus kosoi, Lactobacillus kullabergensis, Lactobacillus kunkeei, Lactobacillus larvae, Lactobacillus leichmannii, Lactobacillus letivazi, Lactobacillus lindneri, Lactobacillus malefermentans, Lactobacillus mali, Lactobacillus manihotivorans, Lactobacillus mellifer, Lactobacillus mellis, Lactobacillus melliventris, Lactobacillus metriopterae, Lactobacillus micheneri, Lactobacillus mindensis, Lactobacillus mixtipabuli, Lactobacillus mobilis, Lactobacillus modestisalitolerans, Lactobacillus mucosae, Lactobacillus mudanjiangensis, Lactobacillus murinus, Lactobacillus musae, Lactobacillus nagelii, Lactobacillus namurensi, Lactobacillus nantensis, Lactobacillus nasuensis, Lactobacillus nenjiangensis, Lactobacillus nodensis, Lactobacillus nuruki, Lactobacillus odoratitolin, Lactobacillus oeni, Lactobacillus oligofermentans, Lactobacillus oris, Lactobacillus oryzae, Lactobacillus otakiensis, Lactobacillus ozensis, Lactobacillus panis, Lactobacillus panisapium, Lactobacillus pantheris, Lactobacillus parabrevis, Lactobacillus parabuchneri, Lactobacillus paracollinoides, Lactobacillus parafarraginis, Lactobacillus paragasseri, Lactobacillus parakefiri, Lactobacillus paralimentarius, Lactobacillus paraplantarum, Lactobacillus pasteurii, Lactobacillus paucivorans, Lactobacillus pentosiphilus, Lactobacillus pentosus, Lactobacillus perolens, Lactobacillus plajomi, Lactobacillus plantarum, Latobacillus pobuzihii, Lactobacillus pontis, Lactobacillus porci, Lactobacillus porcinae, Lactobacillus psittaci, Lactobacillus quenuiae, Lactobacillus raoultii, Lactobacillus rapi, Lactobacillus rennanquilfy, Lactobacillus rennini, Lactobacillus reuteri, Lactobacillus rhamnosus, Lactobacillus rodentium, Lactobacillus rogosae, Lactobacillus rossiae, Lactobacillus ruminis, Lactobacillus saerimneri, Lactobacillus sakei, Lactobacillus salivarius, Lactobacillus sanfranciscensis, Lactobacillus saniviri, Lactobacillus satsumensis, Lactobacillus secaliphilus, Lactobacillus selangorensis, Lactobacillus senioris, Lactobacillus senmaizukei, Lactobacillus sharpeae, Lactobacillus shenzhenensi, Lactobacillus sicerae, Lactobacillus silagei, Lactobacillus silagincola, Lactobacillus siliginis, Lactobacillus similis, Lactobacillus songhuajiangensis, Lactobacillus spicheri, Lactobacillus sucicola, Lactobacillus suebicus, Lactobacillus sunkii, Lactobacillus taiwanensis, Lactobacillus terrae, Lactobacillus thailandensis, Lactobacillus timberlakei, Lactobacillus timonensis, Lactobacillus tucceti, Lactobacillus ultunensis, Lactobacillus uvarum, Lactobacillus vaccinostercus, Lactobacillus vaginalis, Lactobacillus vermiforme, Lactobacillus versmoldensis, Lactobacillus vespulae, Lactobacillus vini, Lactobacillus wasatchensis, Lactobacillus xiangfangensis, Lactobacillus yonginensis and Lactobacillus zymae.

The present invention envisages compositions that comprise at least two of the following species: Lactobacillus crispatus, Lactobacillus helveticus, Lactobacillus jensenii, Lactobacillus amylovorus, Lactobacillus gallinarum, Lactobacillus vaginalis.

The present invention envisages compositions that comprise at least three of the following species: Lactobacillus crispatus, Lactobacillus helveticus, Lactobacillus jensenii, Lactobacillus amylovorus, Lactobacillus gallinarum, Lactobacillus vaginalis.

The present invention envisages compositions that comprise at least four of the following species: Lactobacillus crispatus, Lactobacillus helveticus, Lactobacillus jensenii, Lactobacillus amylovorus, Lactobacillus gallinarum, Lactobacillus vaginalis.

The present invention envisages compositions that comprise at least five of the following species: Lactobacillus crispatus, Lactobacillus helveticus, Lactobacillus jensenii, Lactobacillus amylovorus, Lactobacillus gallinarum, Lactobacillus vaginalis.

The present invention envisages compositions that comprise all of the following species: Lactobacillus crispatus, Lactobacillus helveticus, Lactobacillus jensenii, Lactobacillus amylovorus, Lactobacillus gallinarum, Lactobacillus vaginalis.

In one embodiment, the composition comprises no more than 1, no more than 2, no more than 3, no more than 4, no more than five, no more than six, no more than seven, no more than eight, no more than nine, no more than 10 Lactobacillus species, no more than 15 Lactobacillus species, no more than 20 Lactobacillus species.

In one embodiment, at least 80%, 85%, 90%, 95%, or even 99% of the bacteria of the contemplated compositions are of the genus lactobacillus.

In one embodiment, at least 70%, 75%, 80%, 85%, 90%, 91%, 92 93%, 94%, 95%, 96%, 97%, 98%, 99% of the Lactobacillus bacteria in the composition are of the species crispatus.

In another embodiment, at 70%, 75%, 80%, 85%, 90%, 91 5, 92%, 93%, 94%, 95% of the bacteria in the composition of the species crispatus.

In one embodiment, at least 0.5%, 1%, 2%, 3%, 4%, 5%, 6%, 7%, 8%, 9%, 10% of the Lactobacillus bacteria in the composition are of the species jensenii.

In one embodiment, at least 0.5%, 1%, 2%, 3%, 4%, 5%, 6%, 7%, 8%, 9%, 10% of the bacteria in the composition are of the species jensenii.

Other contemplated bacterial species which may be included in the compositions of the present invention include Mycobacterium sp001665295, Paraburkholderia ginsengiterrae, Colwellia echini, Psychrobacter cibarius, Bacteroides fragilis, Pseudomonas fluorescens, Alcanivorax hongdengensis and GCF-000787395 sp002021095.

Preferably, less than 20%, less than 15%, less than 10%, less than 7.5% less than 5%, less than 2.5%, less than 1% of the bacteria in the composition are of the species Bifidobacterium vaginale. In one embodiment, the composition is devoid of bacteria of the genus Bifidobacterium.

Preferably, less than 20%, less than 15%, less than 10%, less than 7.5% less than 5%, less than 2.5%, less than 1% of the bacteria in the composition are of the species set forth in Table 4. In one embodiment, the composition is devoid of bacteria of the species set forth in Table 4.

In one embodiment, the composition is not a cervicovaginal secretion.

As used herein, the phrase “cervicovaginal secretion” refers to the mixture of mucus secreted by the cervix, shed epithelial cells, vaginal transudate, and bacteria found in the vagina of a woman.

It will be appreciated that the bacteria may be isolated from a cervicovaginal secretion, but naturally occurring cervicovaginal secretions (without additional manipulations) are not contemplated.

As mentioned, the composition may comprise agents which are secreted from the above mentioned bacterial genera.

Agents which are secreted from bacteria include metabolites.

The present invention contemplates compositions comprising at least one metabolite, at least two metabolites, at least three metabolites, four metabolites, five metabolites, 10 metabolites, 20 metabolites, 50 metabolites, 100 metabolites of the above described bacteria.

As used herein, a “metabolite” is an intermediate or product of metabolism. The term metabolite is generally restricted to small molecules and does not include polymeric compounds such as DNA or proteins greater than 100 amino acids in length. A metabolite may serve as a substrate for an enzyme of a metabolic pathway, an intermediate of such a pathway or the product obtained by the metabolic pathway.

According to a particular embodiment, the metabolite is one that alters the composition or function of the vaginal microbiome.

In preferred embodiments, metabolites include but are not limited to sugars, organic acids, amino acids, fatty acids, hormones, vitamins, as well as ionic fragments thereof. In another embodiment, the metabolite is an oligopeptides (less than about 100 amino acids in length).

In particular, the metabolites are less than about 3000 Daltons in molecular weight, and more particularly from about 50 to about 3000 Daltons.

It will be appreciated that the metabolite may be present in the microbes of the composition or secreted from the microbes of the composition (i.e. are obtained by culturing the microbes and collecting the medium, a conditioned medium).

The metabolite may be a primary metabolite (i.e. essential to the microbe for growth) or a secondary metabolite (one that does not play a role in growth, development or reproduction, and is formed during the end or near the stationary phase of growth.

Representative examples of metabolic pathways in which the metabolites of the present invention may be involved include, without limitation, citric acid cycle, respiratory chain, photosynthesis, photorespiration, glycolysis, gluconeogenesis, hexose monophosphate pathway, oxidative pentose phosphate pathway, production and β-oxidation of fatty acids, urea cycle, amino acid biosynthesis pathways, protein degradation pathways such as proteasomal degradation, amino acid degrading pathways, biosynthesis or degradation of: lipids, polyketides (including, e.g., flavonoids and isoflavonoids), isoprenoids (including, e.g., terpenes, sterols, steroids, carotenoids, xanthophylls), carbohydrates, phenylpropanoids and derivatives, alkaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs.

In one embodiment, the compositions comprise agents which are secreted from the above disclosed bacteria and does not comprise the bacteria itself (i.e. a conditioned medium).

The compositions of the present invention may be pH adjusted. They may be packaged into single dosage units for ease of administration. Typically these would be in a dispenser or applicator which has a tip for insertion into the vagina, and a plunger to expel the packaged formulation.

In other embodiments, the bacterial compositions can be lyophilized or spray dried and stored frozen or in a sterile container, for resuspension immediately prior to use. The bacterial compositions can be resuspended with sterile water, a weak acidic solution, gel, or buffer.

In yet another embodiment, the spray dried formulation can be formulated as a disk or wafer, which is inserted into the vagina where it hydrates and repopulates the vaginal mucosa.

In all of these embodiments, dyes, perfumes, pH buffering agents, drying or resuspending agents, or other materials standard in the probiotic field can be incorporated into the formulations.

As mentioned, the compositions of the present invention are used to treat women having symptomatic bacterial vaginosis.

Thus, according to another aspect of the present invention there is provided a method of treating bacterial vaginosis in a subject in need thereof comprising administering to the subject a therapeutically effective amount of a composition comprising between three and twenty species of bacteria or a secretion thereof, wherein at least 70% of the bacteria of the composition are of the species Lactobacillus crispatus and at least two more of said said species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis, GCF-000787395 sp002021095.

As used herein, the term “bacterial vaginosis” refers to a form of vaginal microbial community alteration, with the overgrowth of one of several non-Lactobacillus types of bacteria normally present in the vagina, upsetting the natural balance of vaginal bacteria.

Bacterial vaginosis may be diagnosed on the basis of Amsel's criteria, which requires three of the four following symptoms or signs: homogeneous, thin, white discharge; pH >4.5; a fishy odor of vaginal discharge before or after addition of 10% KOH (i.e., the whiff test); and >20% vaginal epithelial cells studded with adherent coccobacilli (clue cells) on microscopic examination.

In one embodiment, the bacterial vaginosis is intractable—defined as 2, 3, or 4 or more symptomatic episodes of BV during the previous year, or relapsing following repeated antibiotic attempts.

The subject is a woman. In one embodiment, the subject is Caucasian, e.g. of European origin. In another embodiment, the subject is Afro American, black, Hispanic, or African.

In one embodiment, the recipient may also receive daily vaginal treatment with a food acid such as a lactic acid gel, spray or powder before and/or after transplantation to encourage growth of the above disclosed bacteria. Daily treatment may occur for up to 1 week before and/or after transplantation. The preferred concentration range of lactic acid to promote Lactobacillus survival is 1-1.5% lactic acid. Lactic acid is preferred to other types of food acid such as vinegar, lemon juice, and acetic acid, although these may also be utilized.

The subject may be treated with an antibiotic (e.g. intravaginal antibiotic) prior to treatment with the bacterial composition. The antibiotic may be provided for at least 2 days, at least 3 days, at least 4 days, at least 5 days, at least 6 days, at least seven days or longer.

According to another aspect of the present invention there is provided a method of analyzing whether a composition is effective for treating a subject having BV, the method comprising:

(a) providing the composition to the subject; and

(b) analyzing the amount of at least one bacterial species in the vagina of the subject, said bacterial species being selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis and GCF-000787395 sp002021095, wherein an increase in the amount of said bacterial species following said providing as compared to the amount of said bacterial species prior to said providing is indicative that the composition was effective at treating the subject.

Analyzing the amount of any of the above disclosed species may be effected by determining one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

16S and 18S rRNA gene sequences encode small subunit components of prokaryotic and eukaryotic ribosomes respectively. rRNA genes are particularly useful in distinguishing between types of microbes because, although sequences of these genes differ between microbial species, the genes have highly conserved regions for primer binding. This specificity between conserved primer binding regions allows the rRNA genes of many different types of microbes to be amplified with a single set of primers and then to be distinguished by amplified sequences.

In some embodiments, a microbiota sample (e.g. vaginal sample) is directly assayed for a level or set of levels of one or more DNA sequences. In some embodiments, DNA is isolated from a microbiota vaginal sample and isolated DNA is assayed for a level or set of levels of one or more DNA sequences. Methods of isolating microbial DNA are well known in the art. Examples include but are not limited to phenol-chloroform extraction and a wide variety of commercially available kits, including QIAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR) and then sequencing. In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a level or set of levels of one or more types of microbes comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis.

Methods for sequence determination are generally known to the person skilled in the art. Preferred sequencing methods are next generation sequencing methods or parallel high throughput sequencing methods. For example, a bacterial genomic sequence may be obtained by using Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods.

According to one embodiment, the sequencing method allows for quantitating the amount of microbes—e.g. by deep sequencing such as Illumina deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

In some embodiments, determining a level or set of levels of one or more types of microbes comprises determining a level or set of levels of one or more microbial polypeptides. Methods of quantifying polypeptide levels are well known in the art and include but are not limited to Western analysis and mass spectrometry.

As mentioned herein above, as well as (or instead of) analyzing the level of microbes, the present invention also contemplates analyzing the level of microbial products.

Examples of microbial products include, but are not limited to mRNAs, polypeptides, carbohydrates and metabolites.

In some embodiments, the presence, level, and/or activity of metabolites of at least ten species of microbes are measured. In other embodiments, the presence, level, and/or activity of metabolites of between 5 and 50 species of microbes are measured. In other embodiments, the presence, level, and/or activity of metabolites of between 5 and 20 species of microbes are measured. In other embodiments, the presence, level, and/or activity of metabolites of between 5 and 100 species of microbes are measured. In some embodiments, the presence, level, and/or activity of metabolites of between 100 and 1000 or more species of microbes are measured. In other embodiments, the presence, level, and/or activity of metabolites of all bacteria within the microbiome are analyzed. In other embodiments, the presence, level, and/or activity of metabolites of all microbes within the microbiome are measured.

As mentioned, an increase in the amount of at least one, two, three, four, five or all of the of the disclosed species in the vaginal sample following the transplantation as compared to the amount of the same species in the vaginal sample prior to the transplantation is indicative that the composition was effective at treating the subject. In one embodiment, the increase is at least 10% increase, 20% increase, 30% increase, 40% increase, 50% increase or greater.

The analysis may be taken 1 day, 2 days, 3 days, 4 days, five days, six days, 1 week, 2 weeks, 3 weeks, 4 weeks, more than 1 month, more than 2 months, more than 3 months, more than 5 months, more than 6 months following the transplant.

The amount of Bifidobacterium vaginale species may also be analyzed prior to and following transplantation. A decrease in the amount of Bifidobacterium vaginale is preferably by least a 10% decrease, 20% decrease, 30% decrease, 40% decrease, 50% decrease or greater is indicative that the transplantation was effective at treating the BV.

As used herein the term “about” refers to ±10% .

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

### Human Study Cohort

The study was conducted at the Hadassah Medical Center in Jerusalem. AN participants provided written informed consent. Clinical Trial Registration: ClinicalTrials.gov NCT02236429. Recipients' Inclusion criteria: ages 18-50 with symptomatic, recurrent BY, defined as ≥4 symptomatic episodes of BV during the previous year, o required maintenance antibiotic treatment (twice weekly) in order to remain symptom-free, or if they experienced recurrence of BV in ≤2 months following treatment, with a documented history of recurrent BV. Recipients' Exclusion criteria: pregnancy or a planned pregnancy in the upcoming year, infection with Hepatitis B, Hepatitis C, HIV and syphilis.

Donors' inclusion criteria: Ages 18-50, pre-menopausal. Donors' exclusion criteria: history of BV in the last 5 years or any history of recurrent BY, presence of cervico-vaginal STD (Chlamydia trachomatis, Neisseria gonorrhea, Mycoplasma genitalium and Trichomonas vaginalis), positive HPV-testing, vaginal presence of streptococci groups A, C, G, history of recurrent candida vulvovaginitis, history of recurrent urinary tract infections, use of any antibiotics in the month proceeding vaginal fluid collection, use of systemic medication, use of probiotics (orally or vaginally), consumption of herbal or homeopathic remedies, acute illness, history of cancer, history of anogenital dysplasia, history of anogenital HPV, history of anogenital herpes, vulvar or vaginal disease (acute or chronic), pregnancy, abnormal urinalysis or infection, or seropositivity to HIV, hepatitis C, Hepatitis B, herpes or syphilis.

### 16Sequencing and Analysis

For 16S amplicon sequencing, PCR amplification was performed of the 16S rDNA gene and subsequently sequenced using 500 bp paired-end sequencing (Illumina MiSeq). Amplicons spanning the variable region 4 (V4) of the 16S rDNA gene were generated using the appropriate barcoded primers. The reads were then processed using the QIIME 2.2019-1 pipeline. In brief, fasta quality files and a mapping file indicating the barcode sequence corresponding to each sample were used as inputs, reads were split by samples according to the barcode, and taxonomical classification was performed using the Greengenes database (97% OTU identity). Rarefaction was used to exclude samples with insufficient count of reads per sample. For beta-diversity, UniFrac measurements were plotted based on 70,000 reads per sample.

### Shotgun Metagenomics Sequencing and Analysis

Genomic DNA was purified using PowerMag Soil DNA isolation kit (Qiagen) optimized for Tecan automated platform. For shotgun sequencing, Illumina libraries were prepared using Nextera DNA S amp Prep kit (Illumina, FC-121-1031), according to manufacture protocol and sequenced on the Illumina NextSeq platform with a read length of 80 bp. Host reads were removed using KneadData and the hg19 reference. Taxonomic assignment was done using Kraken2 [35] with rarefaction to 100 k reads (species and genus levels). On top, we filtered all the bacteria which their total abundance after rarefaction was <10⁻⁴.

Functional annotation was done using Humann2 [36] pipeline, on the trimmed and host removed KneadData fastq files. The counts were normalized to CPM, combined and annotated to KEGG and GO terms using Humann's built in scripts. These datasets were later filtered from all genes and terms that their abundance was <10⁻³.

### Statistical Analysis

PCA was performed using scikit-learn package in python, as was all the analysis, after performing a log transformation in all cases. The 20 leading loadings (in absolute value) for the first and second principal components were taken.

Kmeans clustering was also done using scikit-learn, for 100 iterations with random_state=the number of iteration.

Permutation tests were performed in the following matter: Under the null hypothesis that the microbiome profiles of samples originating from both groups have the same distribution, μ₀ denote the mean of pairwise dissimilarity between the original groups. For i=1 . . . 10\acute{ }5, the labels of the groups stratifying the permutations are shuffled so that labels are switched only within the same subject's samples. The mean of pairwise dissimilarity of the relabeled groups is denoted by μᵢ. The probability of the null hypothesis to hold is

$$= \frac{\# {\{ {μ}_{i} \geq {μ}_{0} \}}_{i = 1}^{1 {0}^{5}}}{1 {0}^{5}} .$$

I.e. each iteration that had a higher mean of pairwise dissimilarity is counted, and the p-value is that sum divided by the number of iterations.

### Results

An experimental protocol was developed (FIG. 1A) for preliminary assessment of the vaginal microbiome harvest and transplantation from healthy donors into a group of severe and recurrent BV patients. As a first proof of concept, an open-label preliminary trial was conducted including five women aged 27-47 (patients A-E) whose intractable BV was defined as 4 or more symptomatic episodes of BV during the previous year, relapsing following repeated antibiotic attempts [15]. In all of them, multiple different treatment regimens were previously attempted, including twice-weekly maintenance antimicrobial therapy for several months, with relapse of symptomatic BV shortly after each treatment attempt ended, requiring continuous maintenance antibiotic treatment in order to remain symptoms' free. All patients reported a significant negative impact of BV symptoms on their quality of life, including devastating consequences on their relationships, sexual intimacy, and self-esteem.

All five patients were otherwise healthy. Study candidates underwent screening for cervicovaginal infection with C. trachomatis, N. gonorrhea, Mycoplasma genitalium, and T. vaginalis, using a polymerase chain reaction (PCR) assay. Any patient presenting a positive result for any of these infections received the standard recommended treatment [15], with a documented negative assay result deemed mandatory for inclusion in the study. All patients underwent a cervical cytology screening test (Pap test) and PCR-based screening for human papilloma virus (HPV). In case of an abnormal cytology test or positive HPV testing, patients were referred for colposcopy. In addition, vaginal cultures for yeast and bacteria (Streptococci Groups A,B,C and G), urine cultures, urinalysis, and serology analysis for HIV, Hepatitis A, B and C, Treponema pallidum, Herpes viruses, and CMV were performed in all cases.

All patients underwent a thorough and detailed consent process, including a detailed description of foreseen or unforeseen hazards potentially mitigated by this experimental approach. This included a highly detailed explanation of the study procedures, and the limited ability to completely predict future complications as well as possible transfer of infectious agents, including those that cannot be screened for, as well as the very unlikely but non-negligible risk of inadvertent sperm transfer.

BV was diagnosed by the use of Amsel's criteria, which requires three of the four following symptoms or signs: homogeneous, thin, white discharge; pH >4.5; a fishy odor of vaginal discharge before or after addition of 10% KOH (i.e., the whiff test); and >20% vaginal epithelial cells studded with adherent coccobacilli (clue cells) on microscopic examination. On microscopy, bacterial microbiome appearance was defined as “normal” (lactobacilli dominated), “BV” (coccid-bacillary dominated), or “intermediate” [25], as quantified by using the Hay-Ison criteria [26]. At each appointment, including immediately before transplantation, patients underwent a gynecological examination that included an evaluation by Amsel's criteria and microscopy of vaginal discharge.

Donor selection and screening are detailed herein above. The three donors were pre-menopausal, healthy volunteers, aged 35-48 (donors 1-3), who denied a history of BV in the last 5 years or any history of recurrent BV. They were screened using a questionnaire addressing risk factors for potentially transmissible infections. All underwent a PCR-based screening for cervicovaginal C. trachomatis, N. gonorrhea, Mycoplasma genitalium and T. vaginalis. Women exhibiting a positive result were excluded from the study. All donors underwent a cervical cytology screening test and PCR-based screening for HPV, vaginal cultures for yeast and streptococci A,B,C and G, urine cultures, urinalysis and serology analysis for HIV, Hepatitis A, B and C, T. pallidum, Herpes viruses and CMV. According to the protocol, donors were required to either be engaged in monogamous relationships for at least 6 months prior to the study initiation or reported not having intercourse during the same time frame. Donors were requested to abstain from sexual intercourse at least one week before vaginal fluid collection. Additionally, they were requested to avoid douching or bathing (in a swimming pool, hot tub or bath) for at least 48 hours prior to vaginal fluid collection, avoid use of spermicides, diaphragm or condoms for at least one week, and avoid intravaginal medications or topical genital preparations (antifungals, topical steroids etc.) for at least one month prior to sample collection. In case of a recipient CMV seronegative or a negative group B Streptococcus (GBS) status, VMT was received from a CMV seronegative donor or from a negative GBS negative donor, respectively.

Before transplantation, all patients were treated with intravaginal antibiotics [15]. Available treatments included Clindamycin cream 2%, one full applicator (5 g) intravaginally for 7 days (recipients B, E, C) or Metronidazole gel 0.75%, one full applicator (5 g) intravaginally, once a day for 5 days (A, D). In each patient, the chosen antibiotic was the one which previously resulted in a longer symptom-free period. VMT was performed one week after completion of antibiotic treatment [27]. Patients were examined and assessed by Amsel's criteria and microscopy to verify a response to antimicrobial treatment (Table 2).

TABLE 2

| | | Examination- | | amine | | Amsel | microscopic |
|---|---|---|---|---|---|---|---|
| | Symptoms | discharge | pH | Test | microscopy | (out of 4) | diagnosis |
| Recipient A | | | | | | | |
| Before VMT | Discharge, Odor | Yes | 4.6 | positive | 100% clue cells | 4 | BV |
| After Antibiotics-VMT | None | No | 4.6 | negative | no flora | 1 | no flora |
| 7 days post VMT | Discharge | Normal | 4 | negative | lactobacilli, few | 0 | normal |
| 13 days post VMT | Discharge | Normal | 4 | negative | lactobacilli | 0 | Normal |
| 21 days post VMT | Itch | Normal | 4 | negative | lactobacilli | 0 | Normal |
| 56 days post VMT | None | Normal | 4 | negative | lactobacilli | 0 | Normal |
| 96 days post VMT | None | Normal | 4 | negative | lactobacilli | 0 | Normal |
| 138 days post VMT | None | Normal | 4 | negative | lactobacilli | 0 | Normal |
| 167 days post VMT | None | Normal | 4 | negative | lactobacilli | 0 | Normal |
| 230 days post VMT | None | Normal | 4 | negative | lactobacilli | 0 | Normal |
| 293 days post VMT | None | Normal | 4 | negative | lactobacilli | 0 | Normal |
| 356 days post VMT | None | Normal | 4 | negative | lactobacilli | 0 | Normal |
| Recipient B | | | | | | | |
| Before VMT | Discharge, Odor | Yes | 5 | positive | 100% clue cells | 4 | BV |
| After antibiotic- VMT | None | No | 4.6 | negative | No flora | 2 | no flora |
| 7 days post VMT | Discharge | Normal | 4 | negative | lactobacilli | 0 | Normal |
| 14 days post VMT | Less discharge | Normal | 4.6 | negative | lactobacilli | 1 | Normal |
| 21 days post VMT | Less discharge | Normal | 4 | negative | lactobacilli | 0 | Normal |
| 41 days post VMT | None | Normal | 4 | negative | lactobacilli | 0 | Normal |
| 94 days post VMT | None | Normal | 4 | negative | lactobacilli | 0 | Normal |
| 122 days post VMT | None | Normal | 4 | negative | lactobacilli | 0 | Normal |
| 157 days post VMT | None | Normal | 4 | negative | lactobacilli | 0 | Normal |
| Recipient C | | | | | | | |
| Before VMT | Discharge, odor | Yes | 4.7 | positive | 100% clue cells | 4 | BV |
| After Antibiotics- VMT | None | No | 4.5 | negative | No flora | 1 | no flora |
| 7 days post first VMT | None | Normal | 4.6 | negative | Lactobacilli, few | 1 | Intermediate |
| 14 days post first VMT | None | Normal | 4.6 | borderline | Coccobacilli, no clue cells | 2 | BV |
| 21 days post first VMT, Repeated VMT | None | No | 4 | borderline | Coccobacilli, no clue cells | 1 | BV |
| 11 days post second VMT | None | No | 4 | negative | No flora | 0 | No flora |
| 25 days post second VMT | None | No | 5 | negative | No flora | 1 | No flora |
| 43 days post second VMT | None | No | 5 | negative | No flora | 1 | No flora |
| 53 days post second VMT | None | No | 4.6 | negative | Coccobacilli, no clue cells | 1 | BV |
| 100 days post second VMT | None | No | 4.6 | borderline | Coccobacilli, no clue cells | 2 | Intermediate |
| 162 days post second VMT | Odor | No | 4.6 | borderline | Coccobacilli, no clue cells | 2 | BV |
| 217 days post second VMT | Odor | Yes | 5 | positive | Coccobacilli, no clue cells | 3 | BV |
| After Antibiotics- VMT(another donor) | None | No | 4 | negative | No flora | 0 | No flora |
| 25 days post third VMT -donor 3 | None | Normal | 4 | negative | Lactobacilli | 0 | Normal |
| 53 days post third VMT | None | Normal | 4 | negative | Lactobacilli | 0 | Normal |
| 83 days post third VMT | None | Normal | 4 | negative | Lactobacilli | 0 | Normal |
| 119 days post third VMT | None | Normal | 4 | negative | Lactobacilli | 0 | Normal |
| 151 days post third VMT | None | Normal | 4 | negative | Lactobacilli | 0 | Normal |
| 333 days post third VMT | None | Normal | 4 | negative | Lactobacilli | 0 | Normal |
| Recipient D | | | | | | | |
| Before VMT | Discharge, odor | Yes | 5 | positive | 100% clue cells | 4 | BV |
| After Antibiotics- VMT | Odor | No | 5 | negative | no flora | 1 | no flora |
| 14 days post first VMT | End of menstruation | No | 5 | negative | Mixed flora | 1 | intermediate |
| 17 days post first VMT | Discharge, odor | Yes | 5 | positive | Coccobacilli, no clue cells | 3 | BV |
| After Antibiotics- second VMT | None | No | 4.6 | negative | no flora | 1 | no flora |
| 15 days post second VMT | None | No | 4.7 | negative | mixed flora | 1 | intermediate |
| 22 days post second VMT | None | No | 4 | negative | lactobacilli | 0 | Normal |
| 29 days post second VMT | Odor | No | 4.6 | negative | Coccobacilli, no clue cells | 1 | BV |
| 36 days post second VMT | Less discharge | No | 4 | negative | mixed flora | 0 | intermediate |
| 43 days post second VMT | Less discharge | No | 4 | borderline | Coccobacilli, no clue cells | 1 | intermediate |
| After Antibiotics- third VMT | None | No | 4 | negative | no flora | 0 | intermediate |
| 30 days post third VMT | None | Normal | 4 | negative | lactobacilli | 0 | Normal |
| 69 days post third VMT | Odor | Normal | 4 | negative | lactobacilli | 0 | Normal |
| 103 days post third VMT | Odor | Normal | 4 | negative | lactobacilli | 0 | Normal |
| 138 days after third VMT | None | Normal | 4 | negative | lactobacilli | 0 | Normal |
| 159 days after third VMT | None | Normal | 4 | negative | lactobacilli | 0 | Normal |
| 334 days after third VMT | Discharge | Yes | 4 | negative | Coccobacilli, candida | 1 | intermediate |
| 637 days after third VMT | None | Normal | 4 | negative | lactobacilli | 0 | Normal |
| Recipient E | | | | | | | |
| Before VMT | Discharge, Odor | Discharge | 5 | positive | 100% clue cells | 4 | BV |
| After Antibiotics- VMT | None | No | 4.6 | negative | no flora | 1 | No flora |
| 7 days post VMT | Discharge, Odor | Normal | 4 | negative | lactobacilli | 0 | Normal |
| 14 days post VMT | Discharge, Odor | Normal | 4 | negative | lactobacilli | 0 | Normal |
| 28 days post VMT | None | Normal | 4 | negative | lactobacilli | 0 | Normal |
| 36 days post VMT | Odor | Discharge | 4.6 | positive | Coccobacilli, no clue cells | 3 | BV |
| After Antibiotics- second VMT | None | No | 4.6 | negative | no flora | 1 | No flora |
| 21 days after second VMT | Discharge | Normal | 4 | negative | lactobacilli | 0 | Normal |
| 28 days after second VMT | Dischrge, Odor | Discharge | 4 | negative | Coccobacilli, no clue cells | 1 | Intermediate |
| 78 days after second VMT | Discharge | Normal | 4 | negative | Coccobacilli, no clue cells | 0 | Intermediate |
| 108 days after second VMT | Discharge, odor | Normal | 4 | negative | Coccobacilli, no clue cells | 0 | Intermediate |
| 134 days after second VMT | Discharge | Normal | 4 | negative | No flora | 0 | No flora |
| 204 days after second VMT | Discharge | Normal | 4 | negative | Coccobacilli, no clue cells | 0 | Intermediate |

Vaginal fluid for transplantation was collected from the donors starting from the 7^{th} day of the menstrual cycle, using a sterile silicone spatula and taken from the upper half of the vagina and cervical fornices, while avoiding the cervix.

In parallel to VMT sample collection, samples were taken, using the same technique, for molecular analysis, using the ESwab™ Multiple Specimen Collection and Transport System (COPAN) and stored at −80° C. The collected discharge was evaluated by pH and microscopy and diluted with 1 ml sterile saline and transferred with a sterile syringe to the recipient's posterior fornix, without the use of a speculum. VMT was performed at any stage during the cycle of recipient's menstrual cycle, except during menstruation.

Following VMT, recipients were instructed to avoid intercourse for one month, avoid bathing (at a bath, hot tub, swimming pool, etc.) for one week, avoid douching, intravaginal medications and systemic antibiotics for one month and avoid probiotics for the entire follow-up period of one year. According to the protocol, VMT was planned as a single procedure, with an option for repeated VMTs in case of recurrence of symptoms, or appearance of one or more positive Amsel's criteria during follow-up examinations (Table 2, herein above). VMT recipients were evaluated once weekly for the first month, then once every 1-2 months for an additional eleven more months. At each appointment, patients were interviewed and underwent a vaginal examination (including pH measurement, whiff test, and microscopy). Remission of BV was defined at each appointment as disappearance of symptoms, normalization of all Amsel criteria as well as an appearance of normal Lactobacilli-dominated microbiome by light microscopy.

In general, all patients featured long-lasting improvements in their Amsel's scores (FIG. 1B), microscopic vaginal fluid appearance (FIGS. 1C-D), whiff test, discharge, and vaginal fluid pH (FIG. 1D) after 1-3 VMT sessions (Tables 2 and 3).

TABLE 3

| | A | B | C | D | E |
|---|---|---|---|---|---|
| Follow Up avialable (months) | 12 | 4 | 18 | 23 | 8 |
| Pre VMT pH | 4.5 | 5 | 4.7 | 5 | 5 |
| Post VMT pH | 4 | 4 | 4-5 | 4-5 | 4-4.5 |
| Pre VMTdischarge | Yes | Yes | Yes | Yes | Yes |
| Post VMT pH VMTdischarge | Gradual reduction | Gradual reduction | Varies, mostly no | No | Varies, mostly no |
| Pre VMT amine test | Positive | Positive | Yes | Positive | Yes |
| Post VMT amine test | Negative | Negative | Varies, mostly no | Negative | Negative |
| Pre VMT Clue cells | 100% | 100% | 100% | 100% | 100% |
| Post VMT Clue cells | 0 | 0 | 0 | 0 | 0 |
| Pre VMT Total Amsel | 4 | 4 | 4 | 4 | 4 |
| Post VMT Total Amsel | 0 | 0 | 0-3 | 0-1 | 0-3 |
| Pre VMT Hay#Ison score | BV | BV | BV | BV | BV |
| Post VMT Hay#Ison score | Normal | Normal | Varies | Intermediate > | Varies, mostly |
| | | | | Normal | intermediate |
| Antibiotic-vaginal clinamycin | Yes | Yes | Yes | Yes | Yes |
| Antibiotic - vaginal metronidazole | Yes | Yes | Yes | Yes | Yes |
| Contineous maintanance antibiotic | Yes | Yes | Yes | Yes | Yes |
| Previous remission with antibiotic | 2-3 weeks | 4-5 weeks | 3-4 weeks | 4 weeks | 5-7 weeks |
| Antibiotic before VMT | Metronidazole gel | Clindamycin | Clindamycin | Metronidazole gel | Clindamycin |
| Time for relapse (weeks) | 0 | 0 | | 8, than 0 | 5 weeks |
| Donor | 1 | 2 | 1, 3 | 1 | 2 |
| VMT result | Benefitial | Benefitial | after change of | Benefitial | No |
| | | | donor, benefitial | | |

Patients A and B underwent a single VMT each, from donors 1 and 2, respectively. Both reported immediate clinical improvement, with the disappearance of odor within one week after transplantation, and a gradual decrease in discharge, resulting in no symptoms one month after VMT. In both patients, normalization of all Amsel criteria as well as normal lactobacilli-dominated microscopy appearance was documented one week after transplantation and persisted on follow-up examinations (11.5 months in patient A, and 5.5 months in patient B). Patient C received the microbiome of donor 1. She reported an improvement of symptoms following VMT and became BV negative according to Amsel criteria, but her microscopic findings were consistent with persistence of BV. She, therefore, has underwent a repeated VMT from the same donor (1) without preceding antibiotic treatment. For four months the patient reported an improvement of symptoms, and BV was negative according to Amsel criteria. However, 4.5 months following the first VMT she experienced a recurrence of odor, positive Amsel criteria and a “BV” microbiome appearance on microscopy, all consistent with recurring BV. She therefore underwent a third VMT, this time using a sample from a different donor (3) following vaginal antibiotic treatment. Following this VMT, she reported complete resolution of symptoms, Amsel criteria were normalized and microscopy showed a normal Lactobacilli-dominated appearance for 11 months of follow-up. Patient D and E likewise featured a fluctuant course. After a first VMT from donor 1, patient D experienced a recurrence of symptoms, positive Amsel criteria and microscopic findings consistent with BV. She underwent a second VMT from the same donor (1), after which she reported clinical improvement of symptoms and was BV-negative according to Amsel criteria. However, she featured an intermediate vaginal microbial appearance on microscopy and therefore has underwent a third VMT from the same donor (1), after which she reported clinical improvement with the disappearance of odor and improvement of discharge, associated with negative Amsel criteria and a normal Lactobacilli-dominated appearance on microscopy. On evaluation 21 months after the third transplant, the patient reported no recurrences, had negative Amsel criteria and normal microscopy. Following VMT from donor 2, patient E reported a partial symptomatic improvement, associated with negative Amsel's criteria and a normal Lactobacilli-dominated appearance on microscopy, for 4 weeks of follow-up. She then took systemic antibiotics for pharyngitis, and soon after reported a recurrence of odor, accompanied by positive Amsel criteria and BV-characteristic microscopic appearance. She underwent a repeated VMT from the same donor (2), resulting in the normalization of all Amsel criteria, improvement of her microscopic vaginal appearance to an ‘intermediate’ microbiome configuration, coupled with partial symptomatic improvement, for 6.5 months of follow-up.

To characterize the genus-level changes induced by VMT, all donor and recipient vaginal microbiome samples were sequenced using 16s rDNA sequencing. Interestingly, healthy microbiomes clustered differently from the BV diagnosed microbiome (FIGS. 3A-B) after applying principal coordinates analysis (PCoA) with UniFrac distances [28]. Using Bray-Curtis (BC) dissimilarity, the recipients' microbiome was followed before and following VMT. A rapid change in the composition of all microbiome following VMT was observed which correlated with a recovery in all of the Amsel criteria (FIG. 3B).

To study the impact of VMT on vaginal microbiome species-level composition and metagenomic function, all donor and recipient samples underwent shotgun metagenomic sequencing. As expected, donor and recipient microbiomes were found in 2 distinct clusters using principal component analysis (PCA) (FIG. 2A). The impact of VMT on global microbiome composition over the follow-up period was assessed by BC dissimilarity on the species level, as compared to the patients' baseline BV configuration. Four of five VMT recipients exhibited a drastically changed microbiome composition already at the first month following VMT, which correlated with a significant recovery of their Amsel criteria (FIG. 2B), as well as with every discrete clinical criteria (FIG. 4A). Patient C featured the same trend, only after the third and successful VMT (FIG. 2B). One patient's (E) post-VMT microbiome relapsed to her baseline microbiome BV composition following a first VMT failure (FIG. 2B). However, her repeat successful VMT induced a distinctively different configuration, mirrored by a significant species-level BC distances from baseline, similar to the post-VMT trend observed with the other four patients after a successful VMT (FIG. 2B). In four out of the five VMT recipients, the vaginal microbiome configuration remained distinct from the baseline BV configuration over a period of between 5 months to 2 years following a successful VMT (FIG. 2C). Importantly in these patients, the vaginal microbiome composition became similar (yet not identical) to those of their respective donor vaginal microbiome configuration (FIGS. 2C, 2D, 4B). This similarity could be seen after a successful VMT and through the follow-up period (FIG. 2D). Of note, the current preliminary case series is underpowered to statistically test a potential specific donor contribution to a recipient's specific clinical features or microbiome configuration post VMT. A group similarity between the microbiomes of BV patients at baseline, healthy donors at baseline, and a marked shift of all recipients, following VMT, towards the donor cluster was noted (FIG. 2C).

This post-VMT compositional change was mostly dominated by an expansion in members of the Lactobacillus genus, combined with a decrease in members of the Bifidobacterium genus closely related to the Gardenella Genus (The GTDB reference, that was used for taxonomic annotations, classifies Gardnerella genus and Gardnerella vaginalis species as Bifidobacterium and Bifidobacterium vaginale accordingly, FIG. 2E, FIG. 4C). Other genera, including Fannyhessea and Prevotella, were reduced upon successful VMT-induced remission of BV (FIG. 4D). A species-level PCA was used in order to reduce the microbiome complexed dimensionality (FIG. 4E). Indeed, the PCA clustered the samples into “BV” containing mostly samples with one or more Amsel diagnosed BV, and a “healthy” cluster, with mostly no diagnosed clinical features (FIG. 4E). A k-means algorithm (k=2) using the coordinates of the first and second PC, was used in order to define the two clusters that were visually identified (FIG. 4E). The purity score for each Amsel criteria score division was then calculated. In light of the purity scores, the samples were classified into two groups according to their Amsel score, i.e, the first group being samples having Amsel criteria==0 and the second group being all samples with Amsel criteria >0. To see whether the groups are indeed different, a permutational analysis of variance test was conducted using the Bray-Curtis dissimilarity matrix (p-value<0.05). The difference between the two clusters could be explained by the relative levels of Bifidobacterium and Lactobacillus Genera in each sample (FIG. 4F). The most dominant features that contributed to the change in first principle component, that differentiated between the clusters, contained mostly Lactobacillus crispatus specie in the “healthy” cluster, and Bifidobacterium vaginale, which was present in the “BV” cluster (FIG. 2F).

Table 4 is a list of bacteria that is enriched in the BV sample.

TABLE 4

g_Bifidobacterium|s_Bifidobacterium vaginale
g_Fannyhessea|s_Fannyhessea vaginae
g_Bifidobacterium|s_Bifidobacterium vaginale_G
g_Bifidobacterium|s_Bifidobacterium lacrimalis
g_Prevotella|s_Prevotella bivia
g_Bifidobacterium|s_Bifidobacterium vaginale_A
g_Bifidobacterium|s_Bifidobacterium vaginale_D
g_Bifidobacterium|s_Bifidobacterium vaginale_E
g_Aerococcus|s_Aerococcus christensenii
g_Bifidobacterium|s_Bifidobacterium vaginale_C
g_28L|s_28L sp000177555
g_DNF00809|s_DNF00809 sp001552935
g_Dialister|s_Dialister micraerophilus
g_Bifidobacterium|s_Bifidobacterium vaginale_F

Table 5 is a list of bacteria that is enriched in the healthy (donor) sample.

TABLE 5

g_Lactobacillus| s_Lactobacillus crispatus
g_Lactobacillus| s_Lactobacillus helveticus
g_Lactobacillus| s_Lactobacillus jensenii
g_Lactobacillus| s_Lactobacillus amylovorus
g_Lactobacillus| s_Lactobacillus gallinarum
g_Lactobacillus_H| s_Lactobacillus_H vaginalis_A
g_Mycobacterium|s_Mycobacterium sp001665295
g_Paraburkholderia|s_Paraburkholderia ginsengiterrae
g_Colwellials_Colwellia echini
g_Psychrobacter|s_Psychrobacter cibarius
g_Bacteroides|s_Bacteroides fragilis_A
g_Pseudomonas_E|s_Pseudomonas_E fluorescens_AN
g_GCF-000787395|s_GCF-000787395 sp002021095
g_Alcanivorax|s_Alcanivorax hongdengensis

The present inventors next sought to look at the functional microbiome changes that occurred following VMT, using the Kyoto Encyclopedia of Genes and Genomes (KEGG). Interestingly, they observed two distinct functional clusters which separated the “BV” microbiome from the “healthy” one (FIG. 2G), which suited perfectly to the taxonomic differences noted between those conditions (FIG. 2H). Upon recipient follow-up, functional BC distances from the baseline microbiome correlated with a decrease in Amsel criteria (FIG. 5A). Those changes in the functional potential of the microbiome shifted at the time of VMT (FIG. 5B) and remained unaltered over the follow-up period. The identities of these differentially expressed microbiome genes are depicted in FIG. 5C. Similarly to the taxonomical analysis, the current preliminary case series is underpowered to statistically test specific donor and recipient similarities in functional microbiome characteristics, and these can only cluster as BV, healthy donor, and post VMT groups.

Finally, the present inventors performed the same analysis of all pre- and post-VMT samples using Gene Ontology (GO) terms, in which the BC distance from the baseline likewise correlated with the decrease in Amsel criteria (FIG. 6A), although two participants (E and D) appeared not to have changed their GO annotated functional composition according to the BC distances. Nonetheless, a clear PCA cluster could be observed (FIG. 6B) between the “BV” and “healthy” microbiome configurations, and these could be clearly linked to the different taxonomic composition of the BV and healthy clinical states (FIG. 6C). The most dominant GO terms remained stable throughout VMT and the follow-up period, potentially explaining the low BC changes we observed using this analytical method (FIG. 6D). Nonetheless, the second PC did demonstrate a substantial change in GO term signatures over the course of the follow-up period (FIG. 6E).

Collectively, the results suggest a VMT approach as a long-term treatment of recurrent and antibiotics-non-responsive, intractable BV. Four VMT recipients in this preliminary study featured a significant improvement of both clinical symptoms and dysbiotic vaginal microbiome composition and function, which persisted over a long follow-up period. One recipient, whose treatment course was complicated by systemic antibiotics exposure, featured a partial remission. Of note, the 16s rDNA and metagenomic vaginal microbiome profiles accurately reflected the clinical course.

Additionally, this preliminary study highlights (similar to FMT) the potential importance of proper donor-recipient matching, with one VMT recipient failing to show improvement in BV when transplanted from one donor, but entering a long-term remission when transplanted from another Likewise, it suggests that next-generation sequencing of the vaginal microbiome may be developed into a predictive tool assisting in diagnosis, donor and recipient stratification and matching, prognostic assessment and patient follow-up of difficult to treat cases.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. Allsworth, J. E. & Peipert, J. F. Prevalence of bacterial vaginosis: 2001-2004 National Health and Nutrition Examination Survey data. Obstet. Gynecol. (2007). doi:10.1097/01.AOG.0000247627.84791.91
2. Peipert, J. F., Montagno, A. B., Cooper, A. S. & Sung, C. J. Bacterial vaginosis as a risk factor for upper genital tract infection. in American Journal of Obstetrics and Gynecology (1997). doi:10.1016/50002-9378(97)70038-3
3. Babu, G., Singaravelu, B., Srikumar, R., Reddy, S. V. & Kokan, A. Comparative study on the vaginal flora and incidence of asymptomatic vaginosis among healthy women and in women with infertility problems of reproductive age. J. Clin. Diagnostic Res. (2017). doi:10.7860/JCDR/2017/28296.10417
4. Koedooder, R. et al. The vaginal microbiome as a predictor for outcome of in vitro fertilization with or without intracytoplasmic sperm injection: a prospective study. Hum. Reprod. (2019). doi:10.1093/humrep/dez065
5. Haahr, T. et al. Abnormal vaginal microbiota may be associated with poor reproductive outcomes: A prospective study in IVF patients. Hum. Reprod. (2016). doi:10.1093/humrep/dew026
6. Paavonen, J. & Brunham, R. C. Bacterial Vaginosis and Desquamative Inflammatory Vaginitis. N. Engl. J. Med. (2018). doi:10.1056/nejmra1808418
7. Taha, T. E. et al. Bacterial vaginosis and disturbances of vaginal flora: Association with increased acquisition of HIV. AIDS (1998). doi:10.1097/00002030-199813000-00019
8. Joesoef, M. R. & Schmid, G. P. Bacterial vaginosis: Review of treatment options and potential clinical indications for therapy. Clin. Infect. Dis. (1995). doi:10.1093/clinids/20.Supplement_1.S72
9. Marrazzo, J. M. et al. Bacterial vaginosis: Identifying research gaps proceedings of a workshop sponsored by DHHS/NIH/NIAID. Sex. Transm. Dis. (2010). doi:10.1097/OLQ.0b013e318 1 fbbc95
10. Oduyebo, O. O., Anorlu, R. I. & Ogunsola, F. T. The effects of antimicrobial therapy on bacterial vaginosis in non-pregnant women. Cochrane Database of Systematic Reviews (2009). doi:10.1002/14651858.CD006055.pub2
11. Bradshaw, C. S. et al. High Recurrence Rates of Bacterial Vaginosis over the Course of 12 Months after Oral Metronidazole Therapy and Factors Associated with Recurrence. J. Infect. Dis. (2006). doi:10.1086/503780
12. Workowski, K. A., Bolan, G. A. & Centers for Disease Control and Prevention. Sexually transmitted diseases treatment guidelines, 2015. MMWR. Recomm. reports Morb. Mortal. Wkly. report. Recomm. reports (2015).
13. Hay, P. Recurrent bacterial vaginosis. Current Opinion in Infectious Diseases (2009). doi:10.1097/QC0.0b013e32832180c6
14. Sanchez, S., Garcia, P. J., Thomas, K. K., Catlin, M. & Holmes, K. K. Intravaginal metronidazole gel versus metronidazole plus nystatin ovules for bacterial vaginosis: A randomized controlled trial. Am. J. Obstet. Gynecol. (2004). doi:10.1016/j.ajog.2004.06.089
15. Sobel, J. D. et al. Suppressive antibacterial therapy with 0.75% metronidazole vaginal gel to prevent recurrent bacterial vaginosis. Am. J. Obstet. Gynecol. (2006). doi:10.1016/j.ajog.2005.11.041
16. Beigi, R. H., Austin, M. N., Meyn, L. A., Krohn, M. A. & Hillier, S. L. Antimicrobial resistance associated with the treatment of bacterial vaginosis. Am. J. Obstet. Gynecol. (2004). doi:10.1016/j.ajog.2004.05.033
17. Nasioudis, D., Linhares, I. M., Ledger, W. J. & Witkin, S. S. Bacterial vaginosis: a critical analysis of current knowledge. BJOG: An International Journal of Obstetrics and Gynaecology (2017). doi:10.1111/1471-0528.14209
18. Ravel, J. et al. Vaginal microbiome of reproductive-age women. Proc. Natl. Acad. Sci. U.S.A. (2011). doi:10.1073/pnas.1002611107
19. Gajer, P. et al. Temporal dynamics of the human vaginal microbiota. Sci. Transl. Med. (2012). doi:10.1126/scitranslmed.3003605
20. Ravel, J. & Brotman, R. M. Translating the vaginal microbiome: Gaps and challenges. Genome Medicine (2016). doi: 10.1186/s 13073-016-0291-2
21. Swidsinski, A. et al. Adherent biofilms in bacterial vaginosis. Obstet. Gynecol. (2005). doi:10.1097/01.AOG.0000183594.45524.d2
22. Mastromarino, P., Vitali, B. & Mosca, L. Bacterial vaginosis: A review on clinical trials with probiotics. New Microbiologica (2013).
23. Bohbot, J. M. et al. Efficacy and safety of vaginally administered lyophilized Lactobacillus crispatus IP 174178 in the prevention of bacterial vaginosis recurrence. Journal of Gynecology Obstetrics and Human Reproduction (2018). doi: 10.1016/j.jogoh.2017.11.005
24. Malikowski, T., Khanna, S. & Pardi, D. S. Fecal microbiota transplantation for gastrointestinal disorders. Current Opinion in Gastroenterology (2017). doi:10.1097/MOG.0000000000000326
25. Amsel, R. et al. Nonspecific vaginitis. Diagnostic criteria and microbial and epidemiologic associations. Am. J. Med. (1983). doi:10.1016/0002-9343(83)91137-3
26. Ison, C. A. & Hay, P. E. Validation of a simplified grading of Gram stained vaginal smears for use in genitourinary medicine clinics. Sex. Transm. Infect. (2002). doi:10.1136/sti.78.6.413
27. Eriksson, K., Larsson, P. G., Nilsson, M. & Forsum, U. Vaginal retention of locally administered clindamycin. APMIS (2011). doi:10.1111/j.1600-0463.2011.02742.x
28. Lozupone, C. & Knight, R. UniFrac: a new phylogenetic method for comparing microbial communities. Appl. Environ. Microbiol. (2005). doi:10.1128/AEM.71.12.8228-8235.2005
29. Food and Drug Administration. Important Safety Alert Regarding Use of Fecal Microbiota for Transplantation and Risk of Serious Adverse Reactions Due to Transmission of Multi-Drug Resistant Organisms. (2019)
30. Serrano, M. G. et al. Racioethnic diversity in the dynamics of the vaginal microbiome during pregnancy. Nat. Med. 25, 1001-1011 (2019).
31. Fettweis, J. M. et al. The vaginal microbiome and preterm birth. Nat. Med. 25, 1012-1021 (2019).
32. Bolyen, E. et al. Reproducible, interactive, scalable and extensible microbiome data science using QIIME 2. Nat. Biotechnol. 37, 852-857 (2019).
33. Wood, D. E. & Salzberg, S. L. Kraken: ultrafast metagenomic sequence classification using exact alignments. Genome Biol. (2014). doi:10.1186/gb-2014-15-3-r46
34. Méric, G., Wick, R. R., Watts, S. C., Holt, K. E. & Inouye, M. Correcting index databases improves metagenomic studies. bioRxiv 712166 (2019). doi:10.1101/712166
35. Franzosa, E. A. et al. Species-level functional profiling of metagenomes and metatranscriptomes. Nat. Methods (2018). doi:10.1038/s41592-018-0176-y

What is claimed is:

1. A method of treating bacterial vaginosis in a subject in need thereof comprising administering to the subject a therapeutically effective amount of a composition which comprises between three and twenty species of bacteria or a secretion thereof, wherein at least 70% of the bacteria of the composition are of the species Lactobacillus crispatus and at least two more of said species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis and GCF-000787395 sp002021095, thereby treating the bacterial vaginosis.

2. A composition comprising between three and twenty species of bacteria or a secretion thereof, wherein at least 70% of the bacteria of the composition are of the species Lactobacillus crispatus and at least two more of said species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis and GCF-000787395 sp002021095.

3. The method of claim 1, wherein each of said at least two more of said species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis and GCF-000787395 sp002021095.

4. The method of claim 1, wherein said composition comprises a cervico-vaginal secretion.

5. The method of claim 1, wherein said composition comprises a conditioned of said bacteria.

6. The method of claim 1, wherein said at least two more of said species are selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius and Bacteroides fragilis.

7. The method of claim 1, wherein at least 1% of the bacteria are of the species Lactobacillus jensenii.

8. The method of claim 1, wherein less than 10% of the bacteria of the composition are of the species Bifidobacterium vaginale.

9. The method of claim 1, wherein said composition is comprised in a cervico-vaginal secretion from a donor subject who is not suffering from bacterial vaginosis.

10. The method of claim 1, wherein said administering comprises transplanting to the vagina of the subject.

11. The method of claim 1, wherein the bacteria of the composition are lyophilized or spray-dried.

12. The method of claim 1, wherein said subject is Caucasian.

13. The method of claim 1, wherein said bacterial vaginosis is intractable.

14. A method of analyzing whether a composition is effective for treating a subject having BV, the method comprising:
(a) providing the composition to the subject; and
(b) analyzing the amount of at least one bacterial species in the vagina of the subject, said bacterial species being selected from the group consisting of Lactobacillus helveticus, Lactobacillus jensenii, Lactobacillus amylovorus, Lactobacillus gallinarum, Lactobacillus vaginalis, Mycobacterium sp001665295, Paraburkholderia ginsengiterrae, Colwellia echini, Psychrobacter cibarius, Bacteroides fragilis, Pseudomonas fluorescens, Alcanivorax hongdengensis, GCF-000787395 sp002021095, wherein an increase in the amount of said bacterial species following said providing as compared to the amount of said bacterial species prior to said providing is indicative that the composition was effective at treating the subject.

15. The method of claim 14, further comprising analyzing the amount of the bacterial species Bifidobacterium vaginale in the vagina of the subject, wherein a decrease in the amount of said bacterial species Bifidobacterium vaginale following said providing as compared to the amount of said bacterial species Bifidobacterium vaginale prior to said providing is indicative that the composition was effective at treating the subject.

16. The method of claim 14, further comprising analyzing the amount of Lactobacillus crispatus in the vagina of the subject, wherein an increase in the amount of Lactobacillus crispatus following said providing as compared to the amount of said bacterial species prior to said providing is indicative that the composition was effective at treating the subject.


SEQUENCE LISTING (14 sequences)

SEQ ID NO: 1 - DNA, 2906 bp, Artificial sequence
  16S rRNA nucleic acid sequence
tcaagtagaaaagggcgcacggtgaatgcctaggcactaacagccgaagaaggacgtgacgaactacgaaaagcttcggggagcggtaagtacgcagtgatccggagatatccgaatgggggaacccaatgcagagatgcattattgattagtgaatagatagctaatcaaaggaagacgcagtgaactgaaacatctaagtagctgcaggaagagaaagaaagatcgatttccttagtagcggcgagcgaagaggaaagagcccaaaccaagtgatttatcatttggggttgtaggactgcgacgtggcagtgtaagagatagttgaattatctgggaaggtaagccagagagggtgagagccccgtaagcgaaatctcaagcacgcctagcagaatcctgagtaggccgggacacgaggaatcccggttgaaaccgcgaggaccatctcgcaaggctaaatactagttagtgaccgatagtgaaccagtaccgtgagggaaaggtgaaaagaaccccggaaggggagtgaaaaagaacctgaaaccgtgtgtctacaagtagtcaaagcacattaaagtgcaatggcgtgccttttgtagaatgaaccggcgagttacgttatctagcgaggttaagtcagaaaagacggagccggagcgaaagcgagtcttaatagggcgaaaagttaggtgacgtagacccgaaaccaagtgacctacccatgaccaggctgaaggtgtggtaaaacgcactggagggccgaacccacgtaagttaaaaattgcggggatgagttgtgggtagcggtgaaattccaaacgaacttggagatagctggttctctccgaaatagctttagggctagcctcgtggagaggataatggaggtagagctctgtttggactaggggcccgtcaggggttactgaatccagataaactacgaattccagatatccatacacgggagtcagactgcgagtgataagatccgtagtcgaaagggaaacagcccagatcaccagttaaggtccccaaatctatgctaagtggaaaaggatgtggagttgcgtagacaactaggacgttggctcagaagcagccatcattcaaagagtgcgtaatagctcactagtcgagtggcgctgcgccgaaaatttaccggggctaagcatagtaccgaaactgtggatgtgttttaaagagcacgtggtaggagagcgttctaagagcggagaagttgaatcgagaggatcaatggagcgcttagaagtgagaatgccggtatgagtagcgaaagataggtgagaatcctatccgccgaaagactaaggtttcctggggcaggctcgtccgcccagggtaagtcgggacctaaggcaaggccgagaggcgtagtcgatggataacaggtagagattcctgtactgtgtcaaatcgttaagagcgaaggagggacgcaggaggtgaagcacgcatcgcgatggaacgatgttcaagcaacaagtgcggttaagagtcaaatgcttctaaccagcaacacgagttgtgaagaggagtgaattaaaagtagcgaaggtgtggtaatcacactgccaagaaaagcttctagccagagatgacatacccgtaccgcaaaccgacacaggtagtcgagtggagaacactaaggtgagcgagagaactctcgttaaggaactcggcaaaatgaccccgtaacttcggaagaaggggtgctagccaagagattggttagccgcagtgaataggcccaaacaactgtttatcaaaaacacaggtctctgcaaaatcgtaagatgacgtatagaggctgacacctgcccggtgctggaaggttaagaggagagcttagcgtaagcgaaggtttgaattgaagccccagtaaacggcggccgtaactataacggtcctaaggtagcgaaattccttgtcgggtaagttccgacctgcacgaaaggtgtaatgatttgggcactgtctcaacgagagactcggtgaaattataatacccgtgaagatgcgggttacccgcgacaggacggaaagaccccatggagcttcactgtagcttgatattgagtatcttttaaacatgtacaggataggtaggagccagagaagataggacgctagtcttattggaggcaatgttgggatactacccttgtttgaaggatgctctaacctcgacctgtaagccaggccagggacagtgtcaggtgggcagtttgactggggcggtcgcctcctaaagtgtaacggaggcgctcaaaggttccctcagaatggttggaaatcattcgcagagtgtaaaggtataagggagcttgactgcgagagagacaactcgagcaggtaggaaactaggacttagtgatctggtggtaccgtatggaagggccatcactcaacggataaaagctaccctggggataacaggcttatctcccccaagagttcacatcgacggggaggtttggcacctcgatgtcggctcgtcgcatcctggggctgaagtcggtcccaagggttgggctgttcgcccattaaagcggcacgcgagctgggttcagaacgtcgtgagacagttcggtccctatccgtcgtgggcgtaggaaatttgagaggagctgtccttagtacgagaggaccgggatggacgcaccgctggtgtaccagttgttccgccaggagcatcgctgggtagctatgtgcggaagggataagcgctgaaagcatctaagtgcgaagcccccctcaagatgagatttcctttgcgtaagcagtaagacacctcagagacgatgaggtagataggctgggagtggaagtcctgtgaaggatggagcggaccagtactaatcagtcgaggacttgaccaa

SEQ ID NO: 2 - DNA, 2904 bp, Artificial sequence
  16S rRNA nucleic acid sequence
ggtcaagtagaaaagggcgcacggtgaatgcctaggcactaaaagccgatgaaggacgtgacgaactacgaaaagcttcggggagcggtaagtacgcagtgatccggagatgtccgaatgggggaacccaatgcagcgatgcattattggttgatgaataaatagtcaatcaaaggaatacgcagcgaactgaaacatctaagtagctgcaggaagagaaagaaaaatcgatttccttagtagcggcgagcgaagaggaaagagcccaaaccaagtgatttatcatttggggttgtaggactgcaacgtggcagcgtgagtgatagcagaattatctgggaaggtaagccagagagggtgagagccccgtaagcgaaattgcaagcgcgcctagcagaatcctgagtaggccgggacacgagaaatcccggttgaaaccgcgaggaccatctcgcaaggctaaatactaattagtgaccgatagtgaaccagtaccgtgagggaaaggtgaaaagaaccccggaaggggagtgaaatagaacctgaaaccgtgtgtctacaagtagtcaaagcacattaaagtgcaatggcgtgccttttgtagaatgaaccggcgagttacgttatctagcgaggttaagtcagaaaagacggagccggagcgaaagcgagtctgaatagggcgagaagttaggtgacgtagacccgaaaccaagtgacctacccatgaccaggctgaaggcgtggtaaaacgcgctggagggccgaacccacgtaagttaaaaattgcggggatgagttgtgggtagcggtgaaattccaaacgaacttggagatagctggttctctccgaaatagctttagggctagcctcgtggagaggataatggaggtagagctctgtttggactaggggcccgtcaggggttactgaatccagataaactgcgaattccagatatccatacacgggagtcagactgcgagtgataagatccgtagtcgaaagggaaacagcccagatcaccagttaaggtccccaaatctatgctaagtggaaaaggatgtggagttgcgtagacaactaggacgttggctcagaagcagccatcattcaaagagtgcgtaatagctcactagtcgagtgacgctgcgccgaaaatttaccggggctaagcatagtaccgaaactgtggatgtacgtaagtacgtggtaggagagcgttctaagtgcggcgaaggttaaccgagaggataattggagcgcttagaagtgagaatgccggtatgagtagcgaaagataggtgagaatcctatccgccgaaagactaaggtttcctggggcaggctcgtccgcccagggtaagtcgggacctaaggcaaggccgagaggcgtagtcgatggataacaggtagagattcctgtactgtgttcaatcgttaagagcgaaggagggacgcaggaggtgaagcacgcatcgcgatggaacgatgttcaagcgacaagtgcggttaagagtcaaatgcttctaaccagcaacacgagtcgtgatgaggagcgaaataaaagtagcgaaggtgtggtagtcacactgccaagaaaagcttctagccagagagaacatacccgtaccgcaaaccgacacaggtagtcgagtggagaacactaaggtgagcgagagaactctcgttaaggaactcggcaaaatgaccccgtaacttcggaagaaggggtgctggccatgagaatggttagccgcagtgaataggcccaaacaactgtttatcaaaaacacaggtctctgcaaaatcgtaagatgacgtatagaggctgacacctgcccggtgctggaaggttaagaggagagcttagcgtaagcgaaggtttgaattgaagccccagtaaacggcggccgtaactataacggtcctaaggtagcgaaattccttgtcgggtaagttccgacctgcacgaaaggtgtaatgatttgggcactgtctcaacgagagactcggtgaaattataatacccgtgaagatgcgggttacccgcgacaggacggaaagaccccatggagctttactgtagcttgatattgagtatcttttaaacatgtacaggataggtaggagccaaggaaagcaggacgctagttttgctggaggcgatgttgggatactacccttgtttgaaggatgctctaacctcgacctgtaagccaggccagggacagtgtcaggtgggcagtttgactggggcggtcgcctcctaaagtgtaacggaggcgctcaaaggttccctcagaatggttggaaatcattcgcagagtgtaaaggtataagggagcttgactgcgagagaaacagctcgagcaggtaggaaactaggacttagtgatctggtggtaccgcatggaagggccatcactcaacggataaaagctaccctggggataacaggcttatctcccccaagagttcacatcgacggggaggtttggcacctcgatgtcggctcgtcgcatcctggggctgtagtcggtcccaagggttgggctgttcgcccattaaagcggcacgcgagctgggttcagaacgtcgtgagacagttcggtccctatccgtcgtgggcgtaggaaatttgagaggagctgtccttagtacgagaggaccgggatggacgcaccgctggtgtaccagttgttccgccaggagcatcgctgggtagctatgtgcggaagggataagcgctgaaagcatctaagtgcgaagcccccctcaagatgagatttcctttgcgcaagcagtaagacacctcaaagacgatgaggtagataggctgggagtggaagttctgtgaagaatggagcggaccagtactaatcagtcgaggacttgaccaa

SEQ ID NO: 3 - DNA, 1500 bp, Artificial sequence
  16S rRNA nucleic acid sequence
gcaagtcgagcgagcttgcctatagaagttcttcggaatggaaatagatacaagctagcggcggatgggtgagtaacgcgtgggtaacctgcccttaagtctgggataccatttggaaacagatgctaataccggataaaagctactttcgcatgaaagaagtttaaaaggcggcgtaagctgtcgctaaaggatggacctgcgatgcattagctagttggtaaggtaacggcttaccaaggcgatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggattgataagtctgatgtgaaagccttcggctcaaccgaagaactgcatcagaaactgtcaatcttgagtgcagaagaggagagtggaactccatgtgtagcggtggaatgcgtagatatatggaagaacaccagtggcgaaggcggctctctggtctgtaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggttttcccttcggggacaaagagacaggtggtgcatggctgtcgtcagctcgtgtcgtgagatgttgggttaagtcccgcaacgagcgcaacccttgttaatagttgccagcattaagttgggcactctattgagactgccggtgacaaaccggaggaaggtggggatgacgtcaagtcatcatgccccttatgacctgggctacacacgtgctacaatgggcagtacaacgagaagcgaacctgtgaaggcaagcggatctcttaaagctgttctcagttcggactgtaggctgcaactcgcctacacgaagctggaatcgctagtaatcgcggatcagcacgccgcggtgaatacgttcccgggccttgtacacaccgcccgtcacaccatgagagtttgtaacacccaaagtcggtgaggtaacctttggagccagccgcctaaggtgggacagatgattagggtgaagtcgtaacaaggtagccgtaggagaacctgcggttggatcac

SEQ ID NO: 4 - DNA, 2896 bp, Artificial sequence
  16S rRNA nucleic acid sequence
aaagggcgcacggtgaatgcctaggcactaaaagccgatgaaggacgtgacgaactacgaaaagcttcggggagcggtaagtacgctaagatccggagatgtccgaatgggggaacccaatgcagcgatgcattattgaatgatgaatagatagtcattcaaaggaatacgcagtgaactgaaacatctaagtagctgcaggaagagaaagaaaaatcgatttccttagtagcggcgagcgaagaggaaagagcccaaaccaagtgatttatcatttggggttgtaggactgcgacgtggcagcgtgagtgatagcagaattatctgggaaggtaagccagagagggtgagagccccgtaagcgaaattgcaagcgcgcctagcagaatcctgagtaggccgggacacgaggaatcccggttgaaaccgcgaggaccatctcgcaaggctaaatactaattagtgaccgatagtgaaccagtaccgtgagggaaaggtgaaaagaaccccggaaggggagtgaaagagaacctgaaaccgtgtgtctacaagtagtcaaagcacattaaggtgcaatggcgtgccttttgtagaatgaaccggcgagttacgttaactagcgaggttaagtcagaaaagacggagccggagcgaaagcgagtctgaatagggcgagaagttagttgacgtagacccgaaaccaagtgacctacccatgaccaggctgaaggtgtggtaaaacgcactggagggccgaacccacgtaagttaaaaattgcggggatgagttgtgggtagcggtgaaattccaaacgaacttggagatagctggttctctccgaaatagctttagggctagcctcgtggagaggataatggaggtagagctctgtttggactaggggcccgtcaggggttactgaatccagataaactgcgaattccatatatccatacacgggagtcagactgcgagtgataagatccgtagtcgaaagggaaacagcccagatcaccagttaaggtccccaaatctatgctaagtggaaaaggatgtggagttgcgtagacaactaggacgttggctcagaagcagccatcattcaaagagtgcgtaatagctcactagtcgagtggcgctgcgccgaaaatttaccggggctaagcatagtaccgaaactgtggatgtgttttaaagagcacgtggtaggagagcgttctaagtgcggcgaaggctaatcgagaggatagttggagcgcttagaagtgagaatgccggtatgagtagcgaaagataggtgagaatcctatccgccgaaagactaaggtttcctggggcaggctcgtccgcccagggtaagtcgggacctaaggcaaggccgagaggcgtagtcgatggataacaggtagagattcctgtactgtgttcaatcgttatgagcaatggagggacgcaggaggcgaaacacgcatcgagctggatcgatgttcaagcagcgagtgcggttaagagtcaaatgcttctaaccagcaacatgagctgtgatgagtagcgaagtaaaagtagcgaaggtgatgtagtcacactgccaagaaaagcttctagccagagagaacatacccgtaccgcaaaccgacacaggtagtcgagtggagaacactaaggtgagcgagagaactctcgttaaggaactcggcaaaatgaccccgtaacttcggaagaaggggtgctggccatgagagtggttagccgcagtgaataggcccaaacaactgtttatcaaaaacacaggtctctgcaaaatcgtaagatgacgtatagaggctgacacctgcccggtgctggaaggttaagaggagagcttagcgcaagcgaaggtttgaattgaagccccagtaaacggcggccgtaactataacggtcctaaggtagcgaaattccttgtcgggtaagttccgacctgcacgaaaggtgtaatgatttgggcactgtctcaacgagagactcggtgaaattataatacccgtgaagatgcgggttacccgcgacaggacggaaagaccccatggagcttcactgtagcttgatattgagtatcttttaaacatgtacaggataggtaggagccaaggaatgcaggacgctagtcttgcaggaggcaatgttgggatactacccttgtttgaaggatgctctaacctcgacctgtaagccaggccagggacagtgtcaggtgggcagtttgactggggcggtcgcctcctaaagtgtaacggaggcgcccaaaggttccctcagaatggttggaaatcattcgcagagtgtaaaggtataagggagcttgactgcgagagagacaactcgagcagggacgaaagtcgggcttagtgatctggtggtaccgtatggaagggccatcactcaacggataaaagctaccctggggataacaggcttatctcccccaagagttcacatcgacggggaggtttggcacctcgatgtcggctcgtcgcatcctggggctgaagtcggtcccaagggttgggctgttcgcccattaaagcggcacgcgagctgggttcagaacgtcgtgagacagttcggtccctatccgtcgtgggcgtaggaaatttgagaggagctgtccttagtacgagaggaccgggatggacgcaccgctggtgtaccagttgttccgccaggagcatcgctgggtagctatgtgcggaagggataagcgctgaaagcatctaagtgcgaagcccccctcaagatgagatttcctttgcgtaagcagtaagacacctcagagacgatgaggtagataggctgggagtggaagttctgtgaagaatggagcggaccagtactaatcagtcgaggacttgacca

SEQ ID NO: 5 - DNA, 1518 bp, Artificial sequence
  16S rRNA nucleic acid sequence
gcggcgtgcctaatacatgcaagtcgagcgagcagaaccagcagatttacttcggtaatgacgctggggacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcttctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgccatcctaagagattaggagttcccttcggggacgctaagacaggtggtgcatggctgtcgtcagctcgtgtcgtgagatgttgggttaagtcccgcaacgagcgcaacccttgttattagttgccagcattaagttgggcactctaatgagactgccggtgacaaaccggaggaaggtggggatgacgtcaagtcatcatgccccttatgacctgggctacacacgtgctacaatgggcagtacaacgagaagcgagcctgcgaaggcaagcgaatctctgaaagctgttctcagttcggactgcagtctgcaactcgactgcacgaagctggaatcgctagtaatcgcggatcagcacgccgcggtgaatacgttcccgggccttgtacacaccgcccgtcacaccatggaagtctgcaatgcccaaagccggtggcctaaccttcgggaaggagccgtctaaggcagggcagatgactggggtgaagtcgtaacaaggtagccgtaggagaacctgcggttggatca

SEQ ID NO: 6 - DNA, 1488 bp, Artificial sequence
  16S rRNA nucleic acid sequence
  misc_feature (55)..(55): n is a, c, g, t or u
  misc_feature (59)..(59): n is a, c, g, t or u
  misc_feature (79)..(79): n is a, c, g, t or u
  misc_feature (101)..(101): n is a, c, g, t or u
  misc_feature (122)..(122): n is a, c, g, t or u
  misc_feature (124)..(125): n is a, c, g, t or u
  misc_feature (147)..(147): n is a, c, g, t or u
  misc_feature (175)..(175): n is a, c, g, t or u
  misc_feature (183)..(183): n is a, c, g, t or u
  misc_feature (188)..(188): n is a, c, g, t or u
  misc_feature (200)..(200): n is a, c, g, t or u
  misc_feature (202)..(202): n is a, c, g, t or u
  misc_feature (205)..(208): n is a, c, g, t or u
  misc_feature (210)..(211): n is a, c, g, t or u
  misc_feature (214)..(214): n is a, c, g, t or u
  misc_feature (233)..(233): n is a, c, g, t or u
  misc_feature (238)..(238): n is a, c, g, t or u
  misc_feature (256)..(256): n is a, c, g, t or u
  misc_feature (282)..(282): n is a, c, g, t or u
  misc_feature (299)..(299): n is a, c, g, t or u
  misc_feature (328)..(328): n is a, c, g, t or u
  misc_feature (349)..(349): n is a, c, g, t or u
  misc_feature (386)..(386): n is a, c, g, t or u
  misc_feature (412)..(412): n is a, c, g, t or u
  misc_feature (455)..(455): n is a, c, g, t or u
  misc_feature (516)..(516): n is a, c, g, t or u
  misc_feature (522)..(522): n is a, c, g, t or u
  misc_feature (545)..(545): n is a, c, g, t or u
  misc_feature (559)..(559): n is a, c, g, t or u
  misc_feature (575)..(576): n is a, c, g, t or u
  misc_feature (585)..(585): n is a, c, g, t or u
  misc_feature (596)..(596): n is a, c, g, t or u
  misc_feature (622)..(625): n is a, c, g, t or u
  misc_feature (633)..(633): n is a, c, g, t or u
  misc_feature (721)..(721): n is a, c, g, t or u
  misc_feature (750)..(750): n is a, c, g, t or u
  misc_feature (754)..(754): n is a, c, g, t or u
  misc_feature (771)..(771): n is a, c, g, t or u
  misc_feature (793)..(793): n is a, c, g, t or u
  misc_feature (795)..(795): n is a, c, g, t or u
  misc_feature (815)..(815): n is a, c, g, t or u
  misc_feature (827)..(827): n is a, c, g, t or u
  misc_feature (841)..(841): n is a, c, g, t or u
  misc_feature (858)..(858): n is a, c, g, t or u
  misc_feature (864)..(864): n is a, c, g, t or u
  misc_feature (877)..(877): n is a, c, g, t or u
  misc_feature (882)..(882): n is a, c, g, t or u
  misc_feature (889)..(889): n is a, c, g, t or u
  misc_feature (898)..(898): n is a, c, g, t or u
  misc_feature (926)..(926): n is a, c, g, t or u
  misc_feature (938)..(938): n is a, c, g, t or u
  misc_feature (942)..(942): n is a, c, g, t or u
  misc_feature (956)..(956): n is a, c, g, t or u
  misc_feature (965)..(965): n is a, c, g, t or u
  misc_feature (968)..(970): n is a, c, g, t or u
  misc_feature (978)..(979): n is a, c, g, t or u
  misc_feature (1004)..(1004): n is a, c, g, t or u
  misc_feature (1012)..(1013): n is a, c, g, t or u
  misc_feature (1023)..(1023): n is a, c, g, t or u
  misc_feature (1056)..(1056): n is a, c, g, t or u
  misc_feature (1126)..(1126): n is a, c, g, t or u
  misc_feature (1259)..(1259): n is a, c, g, t or u
  misc_feature (1336)..(1336): n is a, c, g, t or u
  misc_feature (1405)..(1405): n is a, c, g, t or u
ttatatgagagtttgatcctggctcaggatgaacgccggcggtgtgcctaatacntgcnagtcgagcgcactggcccanctgatatgacgtgcttgcactnaattgacgggacgggtgagtnanncgtgggcaacctgccctgaagngggggataacatctggaaacaggtgctnataccgcntaacnacgaaaaccacntngtnnnngnntcnaagatggtttcggctatcncttcnggatgggcccgcggtgcnttagctagttggtaaggtaacggctnaccaaggcaatgatgcntagccgagttgagagactgatcggccacnatggaactgagacacggtccntactcctacgggaggcagcagtagggaatcttccacnatgggcgcaagcctgatggagcaacnccgcgtgagtgaagaagggtttcggctcgtaaagctctgttgntggagaagaacgtgcgtgagagtaactgttcacgcagtgacggtatccaaccagaaagtcncggctnactacgtgccagcagccgcggtnatacgtaggtggcnagcgttgtccggattnnttgggcgtnaagcgagcgcnggcggttgcttaggtctgatgtgaannnnttcggctnaaccgaagaagggcatcggaaaccgggcgacttgagtgcagaagaggacagtggaactccatgtgtagcggtggaatgcgtagatatntggaagaacaccagtggcgaaggcggctntctngtctgcaactgacgctnaggctcgaaagcatgggtagcnancaggattagataccctggtngtccatgccgtnaacgatgagtgctnggtgttggagggtttcngccctncagtgccggactnacgcnttaagcnctccgcctngggagtacgaccgcaaggttgaaactcnaaggaattgacngggnccgcacaagcggtngagcatgtngtnnnattcgaanntacgcgaagaaccttaccaggtctngacatctnngcctaacctnagagattaggcgttcttcggggacgcaatgacnggtggtgcatggtcgtcgtcagctcgtgtcgtgagatgttgggttaagtcccgcaacgagcgcaaccctngttactagttgccagcattcagttgggcactctagtgagactgccggtgacaaaccggaggaaggtggggacgacgtcagatcatcatgccccttatgacctgggctacacacgtgctacaatggacggtacnacgagttgcgaactcgcgagagtaagctaatctcttaaagccgttctcagttcggactgtaggctgcaactcgcctncacgaagtcggaatcgctagtaatcgcggatcagcatgccgcggtgaatacgttcccgggccttgtacncaccgcccgtcacaccatggaagtttgcaatgcccaaagtcggtggcctaaccatttggagggaacccctaaggcagggcaga

SEQ ID NO: 7 - DNA, 1557 bp, Artificial sequence
  16S rRNA nucleic acid sequence
ccttagaaaggaggtgatccagccgcaccttccgatacggctaccttgttacgacttcaccccaatcatctatcccaccttaggcggctggctccttacggttacctcaccgacttcgggtgttacaaactctcgtggtgtgacgggcggtgtgtacaaggcccgggaacgtattcaccgcggcgtgctgatccgcgattactagcgattccgacttcatgtaggcgagttgcagcctacaatccgaactgagactggctttaagagattagcttgccgtcaccggcttgcgactcgttgtaccagccattgtagcacgtgtgtagcccaggtcataaggggcatgatgatttgacgtcatccccaccttcctccggtttattaccggcagtctcgctagagtgcccaactcaatgatggcaactaacaataggggttgcgctcgttgcgggacttaacccaacatctcacgacacgagctgacgacaaccatgcaccacctgtcacctctgtcccgaaggaaagctctatctctagagcggtcagagggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctacggcactaaaccccggaaagggtctaacacctagcactcatcgtttacggcgtggactaccagggtatctaatcctgtttgctccccacgctttcgagcctcagcgtcagttacaagccagagagccgctttcgccaccggtgttcctccatatatctacgcatttcaccgctacacatggaattccactctcccctcttgcactcaagttaaacagtttccaaagcgtactatggttaagccacagcctttaacttcagacttatctaaccgcctgcgctcgctttacgcccaataaatccggacaacgctcgggacctacgtattaccgcggctgctggcacgtagttagccgtccctttctggtaagataccgtcacagtgtgaactttccactctcacactcgttcttctcttacaacagagctttacgatccgaaaaccttcttcactcacgcggcgttgctcggtcagacttccgtccattgccgaagattccctactgctgcctcccgtaggagtctgggccgtgtctcagtcccagtgtggccgatcaccctctcaggtcggctatgtatcgtcgccttggtgagccgttaccccaccaactagctaatacaacgcaggtccatctggtagtgatgcaattgcaccttttaagcaaatgtcatgcaacatctactcttatgcggtattagctatcgtttccaatagttatcccccgctaccaggcaggttacctacgcgttactcacccgttcgcaactcatccagaatacggaagcaagctcctccttcagcgttctacttgcatgtattaggcacgccgccagcgttcgtcctgagccaggatcaaactctcattaaa

SEQ ID NO: 8 - DNA, 1453 bp, Artificial sequence
  16S rRNA nucleic acid sequence
gatttctgctcagattgaacgctggcggcatgccttaaacatgcaagtcgaacggcagcacgggagcaatcctggtggcgagtggcgaacgggtgagtaatacatcggaacgtgtcctggagtgggggatagcccggcgaaagccggattaataccgcatacgctctgaggaggaaagggggggatcgcaagacctctcgctcaaggggcggccgatggcagattagctagttggtggggtaaaggcctaccaaggcgacgatctgtagctggtctgagaggacgaccagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaaacttcggggttaatacccctggaggatgacggtaccggaagaataagcaccggctaactacgtgccagcagccgcggtaatacgtagggtgcgagcgttaattcggaattactgggcgtaaagcgtgcgcaggcggtccgctaagaccgatgtgaaatccccgggcttaacctgggaactgcattggtgactggcgggctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgtcgggtcttcattgacttggtaacgtagctaacgcgtgaagttgaccgcctggggagtacggtcgcaagattaaaaactccaaaggaattgacggggacccgcacaagcggtggatgatgtggattaattcgatgcaacgcgaaaaaccttacctaccctttgacatgtatggaatcctgctgagaggtgggagtgcccgaaagggagccataacacaggtgctgcatggctgtcgtcagctcgtgtcgtgagatgttgggttaagtcccgcaacgagcgcaacccttgtccctagttgctacgcaagagcactccagggagactgccggtgacaaaccggaggaaggtggggatgacgtcaagtcctcatggcccttatgggtagggcttcacacgtcatacaatggtcggaacagagggtcgccaagccgcgaggtggagccaatcccagaaaaccgatcgtagtccggatcgcactctgcaactcgagtgcgtgaagctggaatcgctagtaatcgcggatcagcatgccgcggtgaatacgttcccgggtcttgtacacaccgcccgtcacaccatgggagtgggttttaccagaagtggctagtctaaccgcaaggaggaccgtcaccacgtagt

SEQ ID NO: 9 - DNA, 1494 bp, Artificial sequence
  16S rRNA nucleic acid sequence
attgaacgctggcggcaggcttaacacatgcaagtcgagcggtaacaggagaagcttgcttctcgctgacgagcggcggacgggtgagtaatacttaggaatctacctagtagtgggggatagcacggggaaactcgtattaataccgcatacgacctacgggagaaagggggcagtttactgctctcgctattagatgagcctaagtcggattagctagatggtggggtaaaggcctaccatggcgacgatctgtagctggtctgagaggatgatcagccacaccgggactgagacacggcccggactcctacgggaggcagcagtggggaatattggacaatgggggaaaccctgatccagccatgccgcgtgtgtgaagaaggccttttggttgtaaagcactttaagcagtgaagaagactccatggttaatacccatggacgatgacattagctgcagaataagcaccggctaactctgtgccagcagccgcggtaatacagagggtgcaagcgttaatcggaattactgggcgtaaagggagcgtaggtggctctataagtcagatgtgaaatccccgggcttaacctgggaactgcatctgaaactgtagagctagagtatgtgagaggaaggtagaattccaggtgtagcggtgaaatgcgtagagatctggaggaataccgatggcgaaggcagccttctggcataatactgacactgaggctcgaaagcgtgggtagcaaacaggattagataccctggtagtccacgccgtaaacgatgtctactagtcgttgggtcccttgaggacttagtgacgcagctaacgcaataagtagaccgcctggggagtacggccgcaaggttaaaactcaaatgaattgacgggggcccgcacaagcggtggagcatgtggtttaattcgatgcaacgcgaagaaccttacctggtcttgacatatctagaatcctgcagagatgcgggagtgccttcgggaattagaatacaggtgctgcatggctgtcgtcagctcgtgtcgtgagatgttgggttaagtcccgcaacgagcgcaacccttgtccttagttaccagcgggttaagccgggaactctaaggatactgccagtgacaaactggaggaaggcggggacgacgtcaagtcatcatggcccttacgaccagggctacacacgtgctacaatggtaggtacagagggcagctacacagcgatgtgatgcgaatctcaaaaagcctatcgtagtccagattggagtctgcaactcgactccatgaagtaggaatcgctagtaatcgcggatcagaatgccgcggtgaatacgttcccgggccttgtacacaccgcccgtcacaccatgggagttgattgcaccagaagtggatagcttaaccttcgggggagcgttcaccacggtgtggttgatgactggggtgaagtcgtaacaaggtagccgtaggggaacctgc

SEQ ID NO: 10 - DNA, 2878 bp, Artificial sequence
  16S rRNA nucleic acid sequence
aagtaaataagggcgcatggcggatgccttggctctcggaggcgatgaaggacgtgataagctgcgataagcttcgggtaggtgcaaatgacctttgatccgaagatttccgaatgggacaacccggcattctgaaggaatgtcatccaactttgttggaggctaacgcagggaactgaaacatcttagtacctgcaggaaaagaaaataaataatgattcccctagtagtggcgagcgaacggggattagcccaaaccacccatgttacggcatgtgtggggttgtaggaccacgctctcgcaagacagttattgagaagaaacgactggaaagtcgtgccatagacggtgatagcccggtattctaaggtaaccgaagcgtagtggtatcctgagtagcgcggagcacgaggaattctgcgtgaaccagccgggaccatccggtaaggctaaatactcccgagagaccgatagcgaaccagtactgtgaaggaaaggtgaaaagcacttcgaatagaagagtgaaatagtccctgaaaccatgcgcctacaagcggtcggagcagctttagctgtgacggcgtgccttttgcataatgaacctacgagttactttttccggcaaggttaagcatcttgagatgtggatccgaagcgaaagcgagtctgaacagggcggatagtcggaaggagtagacgcgaaaccaagtgatctacccttgggcaggttgaaggttaggtaacactaactggaggaccgaaccgataagcgttgaaaagcttccggatgacctgagggtgggggtgaaaggctaatcaaacttggagatagctcgtactccccgaaatgcatttaggtgcagccttgagagttactaatgtgaggtagagcgactgataagatgcgagggcttcaccgcctatcaagtcttgataaactccgaatgcgcattagttctatctcaggagtgagggcatgggtgctaaggtccatgtcctaaaggagaagaatccagaccatcagctaaggtccccaaataaacactaagttgaactaacgaagtcagattgctaagacagctaggatgttggcttggaagcagccattcatttaaagagtgcgtaacagctcactagtcgaggagtttggcgtggataataatcgggcataagtgttttaccgaagctatgggatcctaatggatcggtaggggagcattccactcagcgttgaaggcgaagcgtgagctttactggagcgtgtggaaaagcaaatgtaggtataagtaacgataaagggggtgagaaaccccctcgccgcaagactaaggtttcctgatcaacgctaatcggatcagggttagtcgggtcctaaggctcagccgaacggtgaggccgatggcagaacaggttaatattcctgtactacctgtaggagtgatgtggagacggaggagtgacaacgccgcggactgacggaatagtccgttaaagggtgtagatgttgattatcccaggcaaatccgggataagagtcgaacctgacagtataccaatttccccggaaaacggtaatagtgcgtgtaaacatactcccaagaaaatccgctaaacttaatcttataggtacccgtaccgtaaacggacacacgtagtcgggttgaatatactcaggcgcttgagtgaatcacggttaaggaactaggcaaattgaccctgtaacttcgggataaagggtccctacccggtgacgggagggcgcagagaataggtccaggcaactgtttaacaaaaacacagggctgtgcaaaattgaaagatcacgtatacagcctgacacctgcccggtgctggaaggttaagaggagatgtcatcgcaagagaagcattgaattgaagccccagtaaacggcggccgtaactataacggtcctaaggtagcgaaattccttgtcgggtaagttccgacctgcacgaatggtgtaatgatctggacactgtctcaaccgtgagctcagtgaaattgtagtatcggtgaagatgccgattacccgcgatgggacgaaaagaccccgtgaacctttactatagcttaacattgaatttgggtaattgatgtgtaggataggccggagactttgaagcaggcacgccagtgtttgtggagtcgctgttgaaatacggccctttggttatttgagttctaactcgctgatgcgaggacactgtttggtgggtagtttgactggggtggtcgcctccaaaagcgtaacggaggcttctaaaggtgccctcacgccgattggtaaccggcggtagagtgtaatggcataagggcgcttgactgggagacttacaagtcgatcaggtaggaaactagagcatagtgatccggtgtttccgtatggaagggacatcgctcaaaggataaaaggtactccggggataacaggctgatccctcccaagagctcatatcgacggaggggtttggcacctcgatgtcggctcgtcacatcctggggctggagaaggtcccaagggttgggctgttcgcccattaaagtggcacgcgagctgggttcagaacgtcgtgagacagttcggtctctatctatcgtgggcgtatgaaatttgcgtggctctgacactagtacgagaggaccgtgttggactgacctctggtttaccggttgtgccgccaggtgcattgccgggtatctaagtcgggattggataagtgctgaaagcatctaagtacgaagccagccacaagattagatttctttcagggtcgttgtagactacaacgttgataggacgcaggtgtacaggtggtaacatcacagccgagcgttactaattgcccgatgacttt

SEQ ID NO: 11 - DNA, 2892 bp, Artificial sequence
  16S rRNA nucleic acid sequence
ggtcaagtgaagaagcgcatacggtggatgccttggcagtcagaggcgatgaaagacgtggtagcctgcgaaaagcttcggggagtcggcaaacagactttgatccggagatgtctgaatgggggaacccagccatcataagatggttatcttgtactgaatacataggtgcaagaggcgaaccaggggaactgaaacatctaagtaccctgaggaaaagaaatcaaccgagattcccttagtagtggcgagcgaacggggactagcccttaagtggctttgagattagcggaacgctctggaaagtgcggccatagtgggtgatagccctgtacgcgaaagtctcttagtcatgaaatcgagtaggacggagcacgagaaactttgtctgaatatggggggaccatcctccaaggctaaatactactgactgaccgatagtgaactagtaccgtgagggaaaggcgaaaagaaccccggagaggggagtgaaatagatcctgaaaccgtatgcgtacaagcagtgggagcagactttgttctgtgactgcgtaccttttgtataatgggtcagcgacttattttcagtggcgagcttaaccgaataggggaggcgtagcgaaagcgagtcttaatagggcgtctagtcgctgggaatagacccgaaaccgggcgatctatccatgggcaggttgaaggttaggtaacactgactggaggaccgaaccgactaccgttgaaaagttagcggatgacctgtggatcggagtgaaaggctaatcaagctcggagatagctggttctcctcgaaagctatttaggtagcgcctcatgtatcactgtagggggtagagcactgtttcggctagggggtcatcccgacttaccaaaccgatgcaaactccgaatacctacaagtgccgagcatgggagacacacggcgggtgctaacgtccgtcgtgaaaagggaaacaacccagaccgtcagctaaggtcccaaagttatggttaagtgggaaacgatgtgggaaggcttagacagctaggaggttggcttagaagcagccaccctttaaagaaagcgtaatagctcactagtcgagtcggcctgcgcggaagatgtaacggggctcaaaccatacaccgaagctacgggtatcacgcaagtgatgcggtagaggagcgttctgtaagcctgtgaaggtgagttgagaagcttgctggaggtatcagaagtgcgaatgctgacatgagtaacgacaatgggtgtgaaaaacacccacgccgaaagaccaaggtttcctgcgcaacgttaatcgacgcagggttagtcggtccctaaggcgaggctgaaaagcgtagtcgatggaaaacaggttaatattcctgtacttctggttattgcgatggagggacggagaaggctaggccagcttggcgttggttgtccaagtttaaggtggtaggctgaaatcttaggtaaatccggggtttcaaggccgagagctgatgacgagttgcctttaggcgacgaagtggttgatgccatgcttccaagaaaagcttctaagcttcagataaccaggaaccgtaccccaaaccgacacaggtggttgggtagagaataccaaggcgcttgagagaactcgggtgaaggaactaggcaaaatggcaccgtaacttcgggagaaggtgcgccggtgagggtgaagcacttgctgcgtaagcccacgccggtcgaagataccaggccgctgcgactgtttattaaaaacacagcactctgcaaacacgaaagtggacgtatagggtgtgacgcctgcccggtgccggaaggttaattgatggggttagctaacgcgaagctcttgatcgaagccccggtaaacggcggccgtaactataacggtcctaaggtagcgaaattccttgtcgggtaagttccgacctgcacgaatggcgtaacgatggcggcgctgtctccacccgagactcagtgaaattgaaatcgctgtgaagatgcagtgtatccgcggctagacggaaagaccccgtgaacctttactatagctttgcactggactttgaatttgcttgtgtaggataggtgggaggctttgaagcgtggacgccagttcgcgtggagccatccttgaaataccaccctggcaactttgaggttctaactcaggtccgttatccggatcgaggacagtgtatggtgggtagtttgactggggcggtctcctcctaaagagtaacggaggagtacgaaggtgcgctcagaccggtcggaaatcggtcgtagagtataaaggcaaaagcgcgcttgactgcgagacagacacgtcgagcaggtacgaaagtaggtcttagtgatccggtggttctgtatggaagggccatcgctcaacggataaaaggtactccggggataacaggctgataccgcccaagagttcatatcgacggcggtgtttggcacctcgatgtcggctcatcacatcctggggctgaagccggtcccaagggtatggctgttcgccatttaaagtggtacgcgagctgggtttagaacgtcgtgagacagttcggtccctatctgccgtggacgtttgagatttgagaggggctgctcctagtacgagaggaccggagtggacgaacctctggtgttccggttgtcacgccagtggcattgccgggtagctatgttcgggaaagataaccgctgaaagcatctaagcgggaaacttgcctcaagatgagatctcactggaaccttgagttccctgaagggccgtcgaagactacgacgttgataggttgggtgtgtaagcgctgtgaggcgttgagctaaccaatactaattgcccgtgaggcttgaccata

SEQ ID NO: 12 - DNA, 1540 bp, Artificial sequence
  16S rRNA nucleic acid sequence
aaactgaagagtttgatcctggctcagattgaacgctggcggtatgcttaacacatgcaagtcgaacgcgaaaataccttcgggtatgagtagagtggcggacgggtgagtaacgcgtaggaatctgcctagtagtgggggacaactaccggaaacggtagctaataccgcatacgccctacgggggaaagcgggggatcttcggacctcgcgctattagatgagcctgcgtcggattagctagttggtagggtaatggcctaccaaggcgacgatccgtagctggtctgagaggatgatcagccacattgggactgagacacggcccaaactcctacgggaggcagcagtggggaatattggacaatgggcgaaagcctgatccagcaataccgcgtgtgtgaagaaggcctgcgggttgtaaagcactttcagtaggaaggaaaagcctcaagttaataactttgggtcttgacgtaacctacagaagaagcaccggctaactccgtgccagcagccgcggtaatacggagggtgcaagcgttaatcggaattactgggcgtaaagcgtacgtaggctgttaggtaagtcagatgtgaaagccccgggctcaacctgggaactgcatttgatactgcctggctagaatctggtagagggtagcggaattccacgtgtagcagtgaaatgcgtagatatgtggaggaacatcagtggcgaaggcggctacctggaccaagattgacgctgaggtacgaaagcgtggggagcaaacgggattagataccccggtagtccacgccgtaaacgatgtcaactagccgttgggttcttttatggacttagtggcgcagctaacgcgataagttgaccgcctggggagtacggtcgcaagattaaaactcaaaggaattgacgggggcccgcacaagcggtggagcatgtggtttaattcgatgcaacgcgaagaaccttaccagctcttgacatcctcagaacttgtcagaaatgacttggtgccttcgggaactgagtgacaggtgctgcatggctgtcgtcagctcgtgtcgtgagatgttgggttaagtcccgtaacgagcgcaacccttgtccctagttgccagcacataatggtgggaactctagggagactgccggtgacaaaccggaggaaggtggggacgacgtcaagtcatcatggcccttacgagctgggctacacacgtgctacaatggtcggtacagagggttgcaaacccgcgagggggagctaatcccagaaaaccggtcgtagtccggattggagtctgcaactcgactccatgaagtcggaatcgctagtaatcgcgaatcagaatgtcgcggtgaatacgttcccgggccttgtacacaccgcccgtcacaccatgggagtgggttgcaccagaagtggctagactaaccttcgggaggtcggttaccacggtgtgattcatgactggggtgaagtcgtaacaaggtagccgtaggggaacctgcggctggatcacctccttaaat

SEQ ID NO: 13 - DNA, 1504 bp, Artificial sequence
  16S rRNA nucleic acid sequence
agagtttgatcctggctcagattgaacgctggcggcaggcctaacacatgcaagtcgagcggaaacgatggtagcttgctaccaggcgtcgagcggcggacgggtgagtaacgcgtgagaatctgcccatttgtgggggataacctggggaaactcaggctaataccgcataatccctacgggggaaagcaggggaccttcgggccttgtgcagatggatgagctcgcgtcggattagcttgttggtgaggtaatggctcaccaaggcgacgatccgtagctggtctgagaggatgatcagccacactgggactgagacacggcccagactcctacgggaggcagcagtggggaatcttggacaatgggggcaaccctgatccagccatgccgcgtgtgtgaagaaggccttcgggttgtaaagcactttcagtagggaggaaggcttagcattaatactgctgagtacttgacgttacctacagaagaagcaccggctaatttcgtgccagcagccgcggtaatacgaaaggtgcgagcgttaatcggaattactgggcgtaaagcgcgcgtaggcggtttgttaagtcagatgtgaaagccccgggctcaacctgggaattgcatttgaaactggcaggctagaatgcagtagagggaggtggaatttccggtgtagcggtgaaatgcgtagagatcggaaggaacaccagtggcgaaggcggcctcctggactgacattgacgctgaggtgcgaaagcgtggggagcaaacaggattagataccctggtagtccacgccgtaaacgatgtctactagtcgttgggaatcttagtattcttggtgacgaagttaacgcgataagtagaccgcctggggagtacggccgcaaggttaaaactcaaatgaattgacgggggcccgcacaagcggtggagcatgtggtttaattcgatgcaacgcgaagaaccttaccaggccttgacatgctgagaactttccagagatggattggtgccttcgggaactcagacacaggtgctgcatggccgtcgtcagctcgtgtcgtgagatgttgggttaagtcccgtaacgagcgcaacccttgtccttagttgccagcacttcgggtgggaactctagggagactgccggtgacaaaccggaggaaggtggggacgacgtcaggtcatcatggcccttacggcctgggctacacacgtgctacaatggacggtacagagggccgcgaagtcgcgaggcggagcaaatcccttaaaaccgttcgtagtccggattggagtctgcaactcgactccatgaagtcggaatcgctagtaatcgcagatcagaatgctgcggtgaatacgttcccgggccttgtacacaccgcccgtcacaccatgggagtggattgcaccagaagtggttagtctaaccttcgggaggacgatcaccacggtgtggttcatgactggggtgaagtcgtaacaaggtagccgt

SEQ ID NO: 14 - DNA, 1510 bp, Artificial sequence
  16S rRNA nucleic acid sequence
ttgaacgctggcggcaggcttaacacatgcaagtcgagcggtaacattcctagcttgctaggagatgacgagcggcggacgggtgagtaatgcttgggaatatgccttatggtgggggacaacagttggaaacgactgctaataccgcataacgtctacggaccaaagggggggatcttcggacctctcgccatttgattagcccaagtgagattagctagttggtaaggtaatggcttaccaaggcgacgatctctagctggtttgagaggatgatcagccacactgggactgagacacggcccagactcctacgggaggcagcagtggggaatattgcacaatgggcgaaagcctgatgcagccatgccgcgtgtgtgaagaaggccttcgggttgtaaagcactttcagtaaggaggaaagggatgtagttaatagctgcattctgtgacgttacttacagaagaagcaccggctaacttcgtgccagcagccgcggtaatacgaggggtgcaagcgttaatcggaattactgggcgtaaagcgttcgtaggcggttatttaagcaagatgtgaaagcccagggctcaaccttggaactgcattttgaactggatgactagagtactgtagagggtggtggaatttccagtgtagcggtgaaatgcgtagagattggaaggaacatcagtggcgaaggcggccacctggacagatactgacgctgaggaacgaaagcgtggggagcgaacaggattagataccctggtagtccacgccgtaaacgatgtcaactagccgtttgtgtccttgagacgtgagtggcgcagctaacgcactaagttgaccgcctggggagtacggccgcaaggttaaaactcaaatgaattgacgggggcccgcacaagcggtggagcatgtggtttaattcgatgcaacgcgaagaaccttaccatcccttgacatccagagaagagactagagatagacttgtgccttcgggaactctgagacaggtgctgcatggctgtcgtcagctcgtgttgtgaaatgttgggttaagtcccgcaacgagcgcaacccctatccttatttgccagcgagttatgtcgggaactctaaggagactgccggtgataaaccggaggaaggtggggacgacgtcaagtcatcatggcccttacgggatgggctacacacgtgctacaatggcaggtacagagggcagcaataccgcgaggtggagcgaatcccagaaagcttgtcgtagtccggattggagtctgcaactcgactccatgaagtcggaatcgctagtaatcgtagatcagaatgctacggtgaatacgttcccgggccttgtacacaccgcccgtcacaccatgggagtgggatgcaaaagaagtggctagtttaaccttcgggaggacggtcaccactttgtgtttcatgactggggtgaagtcgtaacaaggtaaccctaggggaacctggggttggatcacctcc